(19) United States
(12) Patent Application Publication
(10) Pub. No.: US 2019/0060284 A1
(43) Pub. Date: Feb. 28, 2019

(54) PHARMACOLOGICAL MODULATORS OF NAV1.1 VOLTAGE-GATED SODIUM CHANNELS ASSOCIATED WITH MECHANICAL PAIN

(71) Applicant: THE JOHNS HOPKINS UNIVERSITY, Baltimore, MD (US)

(72) Inventor: Frank Bosmans, Annapolis, MD (US)

(21) Appl. No.: 16/080,068

(22) PCT Filed: Feb. 24, 2017
(86) PCT No.: PCT/US2017/019293

§ 371 Date: Aug. 27, 2018

Related U.S. Application Data
(60) Provisional application No. 62/300,237, filed on Feb. 26, 2016.

(51) Int. Cl. A61K 31/4192; A61K 45/06; A61P 25/04; A61P 25/06; A61P 1/00

(52) U.S. Cl. CPC A61K 31/4192; A61K 45/06; A61P 1/00; A61P 25/06; A61P 25/04

(57) ABSTRACT

The present invention provides the use of compounds which selectively block the Nav1.1 subtype of voltage-gated sodium (Nav) channels, whose role in nociception and pain has been unexplored. The present invention demonstrates that Nav1.1-expressing fibers are modality specific nociceptors: their activation elicits robust pain behaviors without neurogenic inflammation and produces profound hypersensitivity to mechanical, but not thermal, stimuli. In the gut, high-threshold mechanosensitive fibers also express Nav1.1 and show enhanced toxin sensitivity in a model of irritable bowel syndrome. The present invention provides an unexpected role for Nav1.1 in regulating the excitability of sensory nerve fibers that underlie mechanical pain, and provides methods of screening for other peptides and small molecules that can modulate Nav1.1 channels and their use in treatment of neurological disorders.

### REFERENCE TO RELATED APPLICATIONS

This application claims the benefit of U.S. Provisional Patent Application No. 62/300,237, filed on Feb. 26, 2016, and is hereby incorporated by reference for all purposes as if fully set forth herein.

### STATEMENT OF GOVERNMENTAL INTEREST

This invention was made with government support under grant no. NS091352, NS065071, NS081907, awarded by the National Institutes of Health. The government has certain rights in the invention.

### INCORPORATION-BY-REFERENCE OF MATERIAL SUBMITTED ELECTRONICALLY

The instant application contains a Sequence Listing which has been submitted in ASCII format via EFS-Web and is hereby incorporated by reference in its entirety. Said ASCII copy, created on Feb. 25, 2015, is named P13939-01_ST25.txt and is 2,364 bytes in size.

### BACKGROUND OF THE INVENTION

Pain is a multimodal system in which functionally distinct classes of primary afferent nerve fibers detect noxious thermal, chemical, and/or mechanical stimuli to elicit protective responses to acute injury as well as maladaptive responses that contribute to persistent pain¹. In these nociceptive neurons, three voltage-gated sodium (Naᵥ) channel subtypes—Naᵥ1.7, Naᵥ1.8 and Naᵥ1.9—have garnered particular attention because mutations affecting these channels are associated with insensitivity to pain or persistent pain syndromes²⁻⁶. Naᵥ1.1 (gene name: SCN1a) is also expressed by somatosensory neurons⁷⁻¹⁰, but no link has been established between this subtype and nociception¹¹. However, mutations affecting Naᵥ1.1 are associated with central nervous system (CNS) disorders such as epilepsy^{12,13,} autism¹⁴, and Alzheimer's¹⁵, and these clinically dominant phenotypes may have masked roles for this subtype in peripheral neurons. For example, gain-of-function mutations in Nadi underlie familial hemiplegic migraine type 3¹⁶, and it is possible that dysfunction of the channel in primary sensory neurons contributes to this pain syndrome, even though this phenotype has been ascribed to a CNS-initiated mechanism¹⁷.

Another major impediment to parsing out roles for Naᵥ1.1 in pain has been a significant challenge in developing subtype-selective pharmacological probes for any member of this highly conserved family of ion channels¹⁸. As such, there still exists an unmet need for identifying molecules capable of acting as subtype-selective pharmacological probes for these receptors and their use in identifying useful modulators of pain.

### SUMMARY OF THE INVENTION

As an alternative to synthetic, small molecule-based discovery platforms, natural products can be exploited as a source of novel agents that have been evolutionarily honed to target their receptors with exquisite specificity. Such agents may be found in complex venoms from spiders, scorpions, cone snails, and snakes, including toxins that excite sensory nociceptors to elicit pain or discomfort in offending predators^{19,20}. The present inventors describe herein two algogenic tarantula toxins that specifically target Naᵥ1.1 and exploit these agents to define a molecular recognition site that achieves Naᵥ1.1 selectivity. Such selectivity enabled the inventors to specifically activate these channels on a subset of myelinated fibers to elicit acute pain and mechanical allodynia, providing new insights into specific roles for Naᵥ1.1 and these sensory nerve fibers in nociception and pain hypersensitivity.

In accordance with an embodiment, the present invention provides a polypeptide having Naᵥ1.1 channel modulating activity.

In accordance with an embodiment, the present invention provides a polypeptide δ-theraphotoxin-Hm1 a (Hm1a) having Naᵥ1.1 channel modulating activity comprising the following amino acid sequence: a) ECRYLFGGCSSTSDCCKHLSCRSDWKYCAWDGTFS (SEQ ID NO: 1); b) a functional fragment of a); c) a functional homolog of a) or b) or functional fragment thereof; and d) a fusion polypeptide comprising an amino acid sequence of any of a) to c).

In accordance with another embodiment, the present invention provides a polypeptide δ-theraphotoxin-Hm1b (Hm1b) having Naᵥ1.1 modulating activity comprising the following amino acid sequence: a) ECRYLFGGCKTTADCCKHLGCRTDLYYCAWDGTF-NH₂ (SEQ ID NO: 2); b) a functional fragment of a); c) a functional homolog of a) or b) or functional fragment thereof; and d) a fusion polypeptide comprising an amino acid sequence of any of a) to c).

In accordance with an embodiment, the present invention provides a nucleic acid sequence encoding any of the polypeptides having Naᵥ1.1 modulating activity or derivatives, homologues, analogues or mimetics thereof as described herein.

In accordance with a further embodiment, the present invention provides a vector comprising one or more nucleic acid sequences encoding any of the polypeptides having Naᵥ1.1 modulating activity or derivatives, homologues, analogues or mimetics thereof as described herein.

In accordance with still another embodiment, the present invention provides a composition comprising one or more polypeptides having Naᵥ1.1 modulating activity or derivatives, homologues, analogues or mimetics thereof as described herein, and at least one or more biologically active agents.

In accordance with another embodiment, the present invention provides a composition comprising one or more polypeptides having Naᵥ1.1 modulating activity or derivatives, homologues, analogues or mimetics thereof as described herein, and at least one or more imaging agents.

In accordance with an embodiment, the present invention provides the use of one or more polypeptides having Naᵥ1.1 modulating activity or derivatives, homologues, analogues or mimetics thereof as described herein, for modulating Naᵥ1.1 receptors in a cell or population of cells expressing the Naᵥ1.1 receptor comprising contacting the cell or population of cells with an effective amount of the polypeptides.

In accordance with another embodiment, the present invention provides the use of a composition comprising one or more polypeptides having Naᵥ1.1 modulating activity or derivatives, homologues, analogues or mimetics thereof as described herein, for modulating Naᵥ1.1 receptors in a subject suffering from a neurological disorder, comprising administering to the subject, an effective amount of a composition comprising one or more polypeptides, and optionally, at least one or more biologically active agents.

In accordance with an embodiment, the present invention provides the use of a composition comprising one or more one or more Naᵥ1.1 channel blockers to inhibit mechanical nociceptors on the myelinated neurons of a subject suffering from a neurological disorder, comprising administering to the subject, an effective amount of a composition comprising one or more Naᵥ1.1 channel blockers and a pharmaceutically acceptable carrier.

In accordance with an embodiment, the present invention provides the use of a composition comprising one or more one or more Naᵥ1.1 channel blockers to inhibit mechanical pain in a subject suffering from a neurological disorder, comprising administering to the subject, an effective amount of a composition comprising one or more Naᵥ1.1 channel blockers and a pharmaceutically acceptable carrier.

In accordance with an embodiment, the present invention provides the use of a composition comprising one or more one or more Naᵥ1.1 channel blockers to inhibit allodynic pain in a subject suffering from a neurological disorder, comprising administering to the subject, an effective amount of a composition comprising one or more Naᵥ1.1 channel blockers and a pharmaceutically acceptable carrier.

In accordance with an embodiment, the present invention provides the use of a composition comprising one or more one or more Naᵥ1.1 channel blockers to inhibit non-inflammatory pain in a subject suffering from a neurological disorder, comprising administering to the subject, an effective amount of a composition comprising one or more Naᵥ1.1 channel blockers and a pharmaceutically acceptable carrier.

In accordance with an embodiment, the present invention provides the use of a composition comprising one or more one or more Naᵥ1.1 channel blockers to inhibit splanchnic colonic afferent neurons of a subject suffering from Irritable Bowel Syndrome (IBS), comprising administering to the subject, an effective amount of a composition comprising one or more Naᵥ1.1 channel blockers and a pharmaceutically acceptable carrier.

In accordance with an embodiment, the present invention provides the use of a composition comprising one or more one or more Naᵥ1.1 channel blockers to treat IBS in a subject suffering from IBS, or pain associated with IBS, comprising administering to the subject, an effective amount of a composition comprising one or more Naᵥ1.1 channel blockers and a pharmaceutically acceptable carrier.

### BRIEF DESCRIPTION OF THE DRAWINGS

FIGS. 1a-1f illustrate that Hm1a selectively targets Naᵥ1.1 in sensory neurons. 1a, The Togo Starburst tarantula, Heteroscodra maculata (image courtesy of Bastian Rast, ArachnoServer database⁵⁰). 1b, Average ratiometric calcium responses from Hm1a-sensitive embryonic rat DRG neurons. Hm1a (500 nM) was applied in the presence or absence of TTX (500 nM), as indicated. For comparison, representative images from identical timepoints are shown for an experiment where TTX is not applied to show persistence of toxin responses (top images). 1c, Representative whole-cell patch clamp recording from Hm1a-sensitive P0 mouse TG neuron. All (15/15) Hm1a responsive neurons (as identified by calcium imaging) displayed similar effect of toxin on sodium current inactivation. Vertical scale bar=1 nA; horizontal scale bar=5 ms. Currents elicited during repeated steps to −30 mV from −90 mV holding potential. 1d, (Left) Average Hm1a-evoked calcium response in the presence of ICA-121431 (500 nM) and after washout (n=11). For comparison, average cellular responses from cells only exposed to Hm1a are shown in grey. (Right) Quantification of maximum calcium signal from Hm1a-responsive cells with or without ICA-121431 (n=25). 1e, Xenopus oocytes expressing cloned human Naᵥ channels were tested for sensitivity to 100 nM Hm1a. Currents in the absence (black) or presence (red) of toxin were monitored during repeated pulses (0.2-1 Hz) to −30 mV (Naᵥ1.1-1.7) or 0 mV (Naᵥ1.8) for 100 ms from a holding potential of −90 mV. Vertical scale bars=100 nA and horizontal scale bars=25 ms. 1f, (Top panels) Representative current clamp recording from mouse TG neuron in the absence (black) or presence (red) of Hm1a (500 nM). (Bottom left) Quantification of action potentials elicited by a 1 s, 20 pA current injection before or after exposure to Hm1a (500 nM, n=4). (Bottom right) Representative action potentials before (black) and after (red) exposure to Hm1a during a 20 pA current injection. Average action potential width significantly increased in the presence of Hm1a by 28.3% ±8.4% (p<0.05, n=4). *p<0.05 and ***p<0.001 based on Student's t-test. Error bars represent mean±SEM.

FIGS. 2a-2c show Hm1a targets S3b-S4 and S1-S2 loops in DIV to inhibit fast inactivation. 2a, Representative traces from oocytes expressing Naᵥ1.1 in the absence (black) and presence (red) of Hm1a (100 nM). Single exponential fits to the inactivation time course are shown in broken lines. Inactivation tau values are plotted (right) showing toxin-induced slowing (**p<0.01, Student's t-test) of inactivation over a range of voltages. 2b, Kᵥ2.1 (far left) and chimeras containing the S3b-S4 motif of each of four hNaᵥ1.1 domains (DI-DIV indicate the domain origin of the transplanted S3b-S4 motif) were tested for sensitivity to Hm1a. Only the DIV chimera displays toxin sensitivity. Currents are shown during 50 ms depolarization to −30 mV. Vertical scale bars=200 nA and horizontal scale bars=10 ms. 2c, rNaᵥ1.4 is insensitive to Hm1a whereas hNaᵥ1.1 is fully sensitive (leftmost traces). Chimeric channels containing S1-S2, S3b-S4, and/or S5-S6 were tested for toxin sensitivity (as indicated). With rNaᵥ1.4 as a backbone, only channels containing the S1-S2 and S3b-S4 regions of Naᵥ1.1 were fully toxin sensitive.

FIGS. 3a-3d show that Naᵥ1.1 is expressed by myelinated, non-C fiber neurons in sensory ganglia. 3a, Representative images showing expression of a variety of cellular markers (left panels) and their overlap with Naᵥ1.1 transcripts (right panels). Markers include immunohistochemical staining for neurofilament 200 (NF200), binding of isolectin B4 (IB4), and in situ histochemistry for TRPV1 or Naᵥ1.7 transcripts, as indicated. Arrows point to cells containing overlapping signal. Asterisks mark non-overlapping cells. 3b, Histogram showing size distribution for all DRG cells (grey bars, 514 cells counted) or Naᵥ1.1-expressing cells (black bars, 324 cells counted). 3c, Quantification of overlap in histological markers. ≥164 cells were counted for each condition from 9-12 independent sections and at least 3 separate mice. 3d, (Left) Representative traces from AM fibers recorded in the skin-nerve preparation showing increased firing following application of 1 μM Hm1 a. (Right) Quantification of firing frequency from AM fibers in the absence (black) or presence (red) of Hm1a (1 μM). Hm1a significantly increases firing in AM fibers in response to all forces tested, which achieves statistical significance at 50 and 100 mN (*** p<0.001 with 2-way ANOVA, # p<0.05 with Bonferonni post-hoc, n=23, 23 and 18 fibers for vehicle and 13, 13 and 10 fibers for Hm1a at 15, 50 and 100 mN forces, respectively).

FIGS. 4a-4f illustrate that Hm1 a elicits non-inflammatory pain and bilateral mechanical allodynia. 4a, Comparison of licking/biting behavior following intraplantar injection (10 μl) of vehicle (PBS) (n=6) versus Hm1a (5 μM) (n=10, **p<0.01). Behavior was unaffected by ablation of TRPV1 fibers (Vlabl, n=5) but significantly reduced in the Per-Cre x Floxed-Naᵥ1.1 (CKO) mice (*p<0.05, n=11). 4b, (Top) Representative histological sections showing c-Fos induction in spinal cord dorsal horn following intraplantar vehicle (PBS) or Hm1a (5 μM) injection. (Bottom) Quantification of c-Fos induction scored as average number of Fos+ cells per dorsal horn section ipsilateral (light grey) or contralateral (dark grey) to injected paw (n=27 sections from 3 mice, ***p<0.001). 4c, Capsaicin- or Hm1a-injected paws (right) next to uninjected contralateral controls (left). (Top right) Relative thickness of injected versus uninjected paws. (Bottom right) Evans blue dye (EBD) extravasation following capsaicin or Hm1a injection (*p<0.05). 4d, Latency of paw withdrawal from noxious heat stimulus (Hargreaves' test) measured after intraplantar injection of vehicle or Hm1a (500 nM). 4e, Mechanical response thresholds (Von Frey filaments) measured in paws ipsilateral (light grey) or contralateral (dark grey) to vehicle (PBS) or toxin (500 nM) injection (n=5 for WT Veh, Vlabl Hm1a and WT Hm1b; n=7 for WT Hm1a; n=9 for CKO Hm1a; **p<0.01, ***p<0.001, ****p<0.0001). 4f, Mechanically-evoked currents were observed from all adult mouse DRG neurons exhibiting sensitivity to Hm1a but not capsaicin (bottom traces, n=10), but not from those sensitive to both Hm1a and capsaicin (top traces, n=15) (stimulus range from 1-9 micron displacement). Kinetic properties of mechanically-evoked currents in Hm1a responders were variable. Error bars represent mean±SEM. P values based on unpaired two-tailed Student's t-test (panels b and c) or one-way ANOVA with post-hoc Tukey's test (panels a,d and e).

FIGS. 5a-5d show that colonic afferents display increased sensitivity to Hm1a in a mouse model of IBS. 5a, (Left) Representative ex vivo single fiber recording from Hm1a (100 nM)-responsive high-threshold fiber from a healthy mouse (arrows indicate application and removal of 2 g von frey hair stimulus). (Middle) Group data showing Hm1a-mediated responses (**p<0.01) from fibers that responded to Hm1a (6/15). Hm1a responders are defined as those in which Hm1a causes 15% increase over baseline. (Right) Group data showing a population (5/10) of healthy, high-threshold mechanoreceptor colonic afferents inhibited by the Naᵥ1.1 blocker ICA-121432 (500 nM, ****p<0.0001). Addition of Hm1a in the presence of ICA-121432 failed to induce mechanical hypersensitivity. 5b, (Left) Representative whole cell current clamp recording of a retrogradely traced colonic DRG neuron in response to 500 ms current injection at rheobase (the minimum current injection required to elicit action potential firing). Recordings were made from the same neuron of a healthy control mouse before and after incubation with Hm1a (10 nM). Horizontal scale bar=250 ms; vertical scale bar=20 mV. (Middle) Group data show a significant reduction in rheobase following Hm1a application in a sub-population (5/11) of neurons (*p<0.05). An Hm1a responsive neuron was defined as exhibiting ≥10% change in rheobase from baseline control. (Right) Hm1a increased the number of action potentials observed at 2× rheobase in these neurons, but not to a level that reached statistical significance. 5c, (Left) High-threshold mechanoreceptive colonic fibers from CVH mice show baseline mechanical hypersensitivity compared with healthy mice, which is further potentiated by Hm1a (100 nM). (Middle) Group data from Hm1a-responsive fibers (4/11) shows a significant increase in mechanically-evoked responses (***p<0.001). (Right) Group data showing a subpopulation of CVH colonic afferents (7/10) inhibited by the Naᵥ1.1 blocker ICA-121432 (500 nM, **p<0.01). The subsequent addition of Hm1a in the presence of ICA-121432 failed to induce mechanical hypersensitivity. 5d, (Left) Example of an Hm1a-responsive colonic DRG neuron in whole-cell current clamp. Addition of Hm1a reduced the rheobase (top traces) and increased action potential firing at 2× rheobase (bottom traces). (Middle) Group data from Hm1a-responsive CVH neurons (7/11) shows Hm1a significantly decreases rheobase (***p<0.001). (Right) In these neurons, Hm1a significantly increased action potential firing at 2x rheobase (**p<0.01, n=7). For comparison, AP firing at 2× rheobase in the presence of Hm1a is shown for both normal and CVH colonic neurons. Hm1a causes significantly more AP firing at 2× rheobase in CVH versus normal Hm1a-responsive neurons (*p<0.05).

FIGS. 6A-6C show Compound B (or FB Naᵥ1.1 blocker)-induced inhibition of mechanosensitivity of a subpopulation of colonic nociceptors from healthy mice. Compound B also prevents Hm1a-induced potentiation of colonic nociceptors.

FIGS. 7A-7C show Compound B (or FB Naᵥ1.1 blocker)-induced inhibition of mechanosensitivity of a subpopulation of colonic nociceptors from mice with chronic visceral hypersensitivity (CVH). Compound B also prevents Hm1a-induced potentiation of colonic nociceptors.

FIG. 8 shows the anti-allodynic effect of Naᵥ1.1 channel blockers of the present invention, such as Compound B vs. vehicle (40% cyclodextrin in saline) on NTG induced mechanical allodynia. Each point represents mean±s.e.m. before, 75 and 120 min after NTG or vehicle administration. 50% Allodynia threshold for all mice tested were statistically similar at baseline. There was no significant difference in allodynia threshold in the NTG/compound B (purple line) and Vehicle/Compound B (red line) groups compared to baseline threshold. Both the NTG/Cyclo (green line) and Vehicle/Cyclo (blue line) groups showed a reduction in mechanical allodynia at the 75 and 120 min time points after NTG/vehicle injection. These results indicate that Compound B was able to reverse NTG induced tactile allodynia and the mixture of Vehicle and cyclodextrin produced an allodynic effect. (* p<0.05, **p<0.01, ***p<0.001, Tukey multiple comparison test using paired samples, n=12 per group).

### DETAILED DESCRIPTION OF THE INVENTION

The present inventors' findings now unambiguously implicate Naᵥ1.1 and Naᵥ1.1-expressing, myelinated afferents in nociception. Activation or sensitization of these fibers is sufficient to elicit robust acute pain, as well as mechanical allodynia, without triggering neurogenic inflammation, distinguishing these fibers from the well-characterized C-nociceptor. Previous studies have implicated myelinated Aδ fibers in mechano-nociception^{44,45,} and Naᵥ1.1 now provides an important new marker with which to more precisely identify the contribution of these fibers to acute and chronic pain.

The present inventors' findings with the CVH model show that pharmacological blockade of Naᵥ1.1 represents a novel therapeutic strategy for diminishing chronic pain associated with IBS, and perhaps other pain conditions associated with mechanical sensitization, including migraine headache. While Naᵥ1.1 activity in the brain may underlie aura in FHM3 patients¹⁷, the present inventions show that these gain-of-function mutations may also produce migraine pain through actions of Naᵥ1.1 in mechanical nociceptors.

In accordance with an embodiment, the present invention provides a polypeptide having Naᵥ1.1 channel modulating activity.

In accordance with an embodiment, the present invention provides a polypeptide δ-theraphotoxin-Hm1 a (Hm1a) having Naᵥ1.1 channel modulating activity comprising the following amino acid sequence: a) ECRYLFGGCSSTSDCCKHLSCRSDWKYCAWDGTFS (SEQ ID NO: 1); b) a functional fragment of a); c) a functional homolog of a) or b) or functional fragment thereof; and d) a fusion polypeptide comprising an amino acid sequence of any of a) to c).

In accordance with another embodiment, the present invention provides a polypeptide δ-theraphotoxin-Hm1b (Hm1b) having Naᵥ1.1 channel modulating activity comprising the following amino acid sequence: a) ECRYLFGGCKTTADCCKHLGCRTDLYYCAWDGTF-NH2 (SEQ ID NO: 2); b) a functional fragment of a); c) a functional homolog of a) or b) or functional fragment thereof; and d) a fusion polypeptide comprising an amino acid sequence of any of a) to c).

In accordance with an embodiment, the present invention provides a nucleic acid sequence encoding any of the polypeptides having Naᵥ1.1 channel modulating activity or derivatives, homologues, analogues or mimetics thereof as described herein.

In accordance with a further embodiment, the present invention provides a vector comprising one or more nucleic acid sequences encoding any of the polypeptides having Naᵥ1.1 channel modulating activity or derivatives, homologues, analogues or mimetics thereof as described herein.

In accordance with still another embodiment, the present invention provides a composition comprising one or more polypeptides having Naᵥ1.1 channel modulating activity or derivatives, homologues, analogues or mimetics thereof as described herein, and at least one or more biologically active agents.

In accordance with another embodiment, the present invention provides a composition comprising one or more polypeptides having Naᵥ1.1 channel modulating activity or derivatives, homologues, analogues or mimetics thereof as described herein, and at least one or more imaging agents.

In accordance with an embodiment, the present invention provides the use of one or more polypeptides having Naᵥ1.1 channel modulating activity or derivatives, homologues, analogues or mimetics thereof as described herein, for modulating Naᵥ1.1 receptors in a cell or population of cells expressing the Naᵥ1.1 receptor comprising contacting the cell or population of cells with an effective amount of the polypeptides.

In accordance with another embodiment, the present invention provides the use of a composition comprising one or more polypeptides having Naᵥ1.1 channel modulating activity or derivatives, homologues, analogues or mimetics thereof as described herein, for modulating Naᵥ1.1 receptors in a subject suffering from a neurological disorder, comprising administering to the subject, an effective amount of a composition comprising one or more polypeptides, and optionally, at least one or more biologically active agents.

The term, “amino acid” includes the residues of the natural a-amino acids (e.g., Ala, Arg, Asn, Asp, Cys, Glu, Gln, Gly, His, Lys, Ile, Leu, Met, Phe, Pro, Ser, Thr, Trp, Tyr, and Val) in D or L form, as well as β-amino acids, synthetic and non-natural amino acids. Many types of amino acid residues are useful in the polypeptides and the invention is not limited to natural, genetically-encoded amino acids. Examples of amino acids that can be utilized in the peptides described herein can be found, for example, in Fasman, 1989, CRC Practical Handbook of Biochemistry and Molecular Biology, CRC Press, Inc., and the reference cited therein. Another source of a wide array of amino acid residues is provided by the website of RSP Amino Acids LLC.

Reference herein to “derivatives” includes parts, fragments and portions of the inventive Naᵥ1.1 channel modulating peptides. A derivative also includes a single or multiple amino acid substitution, deletion and/or addition. Homologues include functionally, structurally or stereochemically similar peptides from venom from the same species of spider or from within the same genus or family of spider. All such homologues are contemplated by the present invention.

Analogs and mimetics include molecules which include molecules which contain non-naturally occurring amino acids or which do not contain amino acids but nevertheless behave functionally the same as the peptide. Natural product screening is one useful strategy for identifying analogs and mimetics.

Examples of incorporating non-natural amino acids and derivatives during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, omithine, sarcosine, 4-amino-3-hydroxy-6-methylheptanoic acid, 2-thienyl alanine and/or D-isomers of amino acids. A partial list of known non-natural amino acid contemplated herein is shown in Table 1.

TABLE 1
Non-natural Amino Acids

| Non-conventional | | Non-conventional | |
|---|---|---|---|
| amino acid | Code | amino acid | Code |
| α-aminobutyric acid | Abu | L-N-methylalanine | Nmala |
| α-amino-a-methylbutyrate | Mgabu | L-N-methylarginine | Nmarg |
| aminocyclopropane- | Cpro | L-N-methylasparagine | Nmasn |
| carboxylate | | L-N-methylaspartic acid | Nmasp |
| aminoisobutyric acid | Aib | L-N-methylcysteine | Nmcys |
| aminonorbomyl- | Norb | L-N-methylglutamine | Nmgln |
| carboxylate | | L-N-methylglutamic acid | Nmglu |
| cyclohexylalanine | | Chexa L-N-methylhistidine | Nmhis |
| cyclopentylalanine | Cpen | L-N-methylisolleucine | Nmile |
| D-alanine | Dal | L-N-methylleucine | Nmleu |
| D-arginine | Darg | L-N-methyllysine | Nmlys |
| D-aspartic acid | Dasp | L-N-methylmethionine | Nmmet |
| D-cysteine | Dcys | L-N-methylnorleucine | Nmnle |
| D-glutamine | Dgln | L-N-methylnorvaline | Nmnva |
| D-glutamic acid | Dglu | L-N-methylornithine | Nmorn |
| D-histidine | Dhis | L-N-methylphenylalanine | Nmphe |
| D-isoleucine | Dile | L-N-methylproline | Nmpro |
| D-leucine | Dleu | L-N-methylserine | Nmser |
| D-lysine | Dlys | L-N-methylthreonine | Nmthr |
| D-methionine | Dmet | L-N-methyltryptophan | Nmtrp |
| D-ornithine | Dorn | L-N-methyltyrosine | Nmtyr |
| D-phenylalanine | Dphe | L-N-methylvaline | Nmval |
| D-proline | Dpro | L-N-methylethylglycine | Nmetg |
| D-serine | Dser | L-N-methyl-t-butylglycine | Nmtbug |
| D-threonine | Dthr | L-norleucine | Nle |
| D-tryptophan | Dtrp | L-norvaline | Nva |
| D-tyrosine | Dtyr | α-methyl-aminoisobutyrate | Maib |
| D-valine | Dval | α-methyl-γ-aminobutyrate | Mgabu |
| D-α-methylalanine | Dmala | α-methylcyclohexylalanine | Mchexa |
| D-α-methylarginine | Dmarg | α-methylcylcopentylalanine | Mcpen |
| D-α-methylasparagine | Dmasn | α-methyl-α-napthylalanine | Manap |
| D-α-methylaspartate | Dmasp | α-methylpenicillamine | Mpen |
| D-α-methylcysteine | Dmcys | N-(4-aminobutyl)glycine | Nglu |
| D-α-methylglutamine | Dmgln | N-(2-aminoethyl)glycine | Naeg |
| D-α-methylhistidine | Dmhis | N-(3-aminopropyl)glycine | Norn |
| D-α-methylisoleucine | Dmile | N-amino-α-methylbutyrate | Nmaabu |
| D-α-methylleucine | Dmleu | α-napthylalanine | Anap |
| D-α-methyllysine | Dmlys | N-benzylglycine | Nphe |
| D-α-methylmethionine | Dmmet | N-(2-carbamylethyl)glycine | Ngln |
| D-α-methylornithine | Dmorn | N-(carbamylmethyl)glycine | Nasn |
| D-α-methylphenylalanine | Dmphe | N-(2-carboxyethyl)glycine | Nglu |
| D-α-methylproline | Dmpro | N-(carboxymethyl)glycine | Nasp |
| D-α-methylserine | Dmser | N-cyclobutylglycine | Ncbut |
| D-α-methylthreonine | Dmthr | N-cycloheptylglycine | Nchep |
| D-α-methyltryptophan | Dmtrp | N-cyclohexylglycine | Nchex |
| D-α-methyltyrosine | Dmty | N-cyclodecylglycine | Ncdec |
| D-α-methylvaline | Dmval | N-cylcododecylglycine | Ncdod |
| D-N-methylalanine | Dnmala | N-cyclooctylglycine | Ncoct |
| D-N-methylarginine | Dnmarg | N-cyclopropylglycine | Ncpro |
| D-N-methylasparagine | Dnmasn | N-cycloundecylglycine | Ncund |
| D-N-methylaspartate | Dnmasp | N-(2,2-diphenylethyl)glycine | Nbhm |
| D-N-methylcysteine | Dnmcys | N-(3,3-diphenylpropyl)glycine | Nbhe |
| D-N-methylglutamine | Dnmgln | N-(3-guanidinopropyl)glycine | Narg |
| D-N-methylglutamate | Dnmglu | N-(1-hydroxyethyl)glycine | Nthr |
| D-N-methylhistidine | Dnmhis | N-(hydroxyethyl))glycine | Nser |
| D-N-methylisoleucine | Dnmile | N-(imidazolylethyl))glycine | Nhis |
| D-N-methylleucine | Dnmleu | N-(3-indolylyethyl)glycine | Nhtrp |
| D-N-methyllysine | Dnmlys | N-methyl-γ-aminobutyrate | Nmgabu |
| N-methylcyclohexylalanine | Nmchexa | D-N-methylmethionine | Dnmmet |
| D-N-methylornithine | Dnmorn | N-methylcyclopentylalanine | Nmcpen |
| N-methylglycine | Nala | D-N-methylphenylalanine | Dnmphe |
| N-methylaminoisobutyrate | Nmaib | D-N-methylproline | Dnmpro |
| N-(1-methylpropyl)glycine | Nile | D-N-methylserine | Dnmser |
| N-(2-methylpropyl)glycine | Nleu | D-N-methylthreonine | Dnmthr |
| D-N-methyltryptophan | Dnmtrp | N-(1-methylethyl)glycine | Nval |
| D-N-methyltyrosine | Dnmtyr | N-methyla-napthylalanine | Nmanap |
| D-N-methylvaline | Dnmval | N-methylpenicillamine | Nmpen |
| γ-aminobutyric acid | Gabu | N-(p-hydroxyphenyl)glycine | Nhtyr |
| L-t-butylglycine | Tbug | N-(thiomethyl)glycine | Ncys |
| L-ethylglycine | Etg | penicillamine | Pen |
| L-homophenylalanine | Hphe | L-α-methylalanine | Mala |
| L-α-methylarginine | Marg | L-α-methylasparagine | Masn |
| L-α-methylaspartate | Masp | L-α-methyl-t-butylglycine | Mtbug |
| L-α-methylcysteine | Mcys | L-methylethylglycine | Metg |
| L-α-methylglutamine | Mgln | L-α-methylglutamate | Mglu |
| L-α-methylhistidine | Mhis | L-α-methylhomophenylalanine | Mhphe |
| L-α-methylisoleucine | Mile | N-(2-methylthioethyl)glycine | Nmet |
| L-α-methylleucine | Mleu | L-α-methyllysine | Mlys |
| L-α-methylmethionine | Mmet | L-α-methylnorleucine | Mnle |
| L-α-methylnorvaline | Mnva | L-α-methylornithine | Morn |
| L-α-methylphenylalanine | Mphe | L-α-methylproline | Mpro |
| L-α-methylserine | Mser | L-α-methylthreonine | Mthr |
| L-α-methyltryptophan | Mtrp | L-α-methyltyrosine | Mtyr |
| L-α-methylvaline | Mval | L-N-methylhomophenylalanine | Nmhphe |
| N-(N-(2,2-diphenylethyl) | Nnbhm | N-(N-(3,3-diphenylpropyl) | Nnbhe |
| carbamylmethyl)glycine | | carbamylmethyl)glycine | |
| 1-carboxy-1-(2,2-diphenyl- | Nmbc | | |
| ethylamino)cyclopropane | | | |

Analogs of the subject peptides contemplated herein include modifications to side chains, incorporation of non-natural amino acids and/or their derivatives during peptide synthesis and the use of crosslinkers and other methods which impose conformational constraints on the peptide molecule or their analogs.

In accordance with an embodiment, the present invention provides a polypeptide δ-theraphotoxin-Hm1 a (Hm1 a) variant having Naᵥ1.1 channel modulating activity comprising the following amino acid sequence: a) ECRYLFGGCSSTSDCCKHLSCRSDWKYCAWDGTF (SEQ ID NO: 3); b) a functional fragment of a); c) a functional homolog of a) or b) or functional fragment thereof; and d) a fusion polypeptide comprising an amino acid sequence of any of a) to c).

In accordance with another embodiment, the present invention provides a polypeptide δ-theraphotoxin-Hm1b (Hm1b) variant having Naᵥ1.1 channel modulating activity comprising the following amino acid sequence: a) ECRYLFGGCKTTADCCKHLGCRTDLYYCAWDGTF (SEQ ID NO: 4); b) a functional fragment of a); c) a functional homolog of a) or b) or functional fragment thereof; and d) a fusion polypeptide comprising an amino acid sequence of any of a) to c).

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitization, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carbethoxylation with diethylpyrocarbonate.

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety such as maleimido or dithio moiety (SH) or carbodiimide (COOH). In addition, peptides can be conformationally constrained by, for example, incorporation of C_{α} and N_{α}-methylamino acids, introduction of double bonds between C_{α} and C_{β} atoms of amino acids and the formation of cyclic peptides or analogues by introducing covalent bonds such as forming an amide bond between the N and C termini, between two side chains or between a side chain and the N or C terminus.

The present invention further contemplates small chemical analogs of the subject peptides capable of acting as antagonists or agonists of the Naᵥ1.1 channel modulating peptides of the present invention. Chemical analogs may not necessarily be derived from the peptides themselves but may share certain conformational similarities. Alternatively, chemical analogs may be specifically designed to mimic certain physiochemical properties of the peptides. Chemical analogs may be chemically synthesized or may be detected following, for example, natural product screening.

For example, the present inventors have previously discovered a new class of Naᵥ1.1 channel blockers, which are derived from rufinamide, and which were disclosed in U.S. Patent Publication No. 2015/0336904, filed Jul. 31, 2015, and incorporated by reference herein as if set forth in its entirety. As such, in accordance with some embodiments, the present invention provides a pharmaceutical composition comprising a compound of formula I:

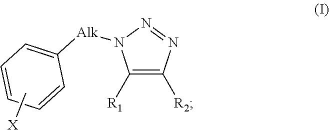

or a salt, solvate, or stereoisomer thereof, wherein X is H, or one or more electron withdrawing groups such as a halogen, NH₂, NO₂, SO₂, CN, or a C₁-C₆ alkyl group; Alk is C₁-C₃ alkyl; R₁ is H, C₁-C₆ alkyl, which may be substituted with OH, NH₂, alkylamino, amido, acyl, sulfonyl, sulfonylamino, and cyano groups; and R₂, is C₁-C₆ alkyl, alkenyl, and phenyl, which may be substituted with one or more OH, NH₂, alkylamino, amido, acyl, carboxyl, methoxyl, sulfonyl, and cyano groups, and a pharmaceutically acceptable carrier, in an effective amount, for use as a medicament, preferably for use in modulating the opening of one or more voltage-gated sodium Naᵥ1.1 channels in one or more neurons of a subject, or for use in treating a Naᵥ1.1 channel associated neurological disorder in a subject.

In some alternative embodiments, the compound of formula I is selected from the group consisting of:

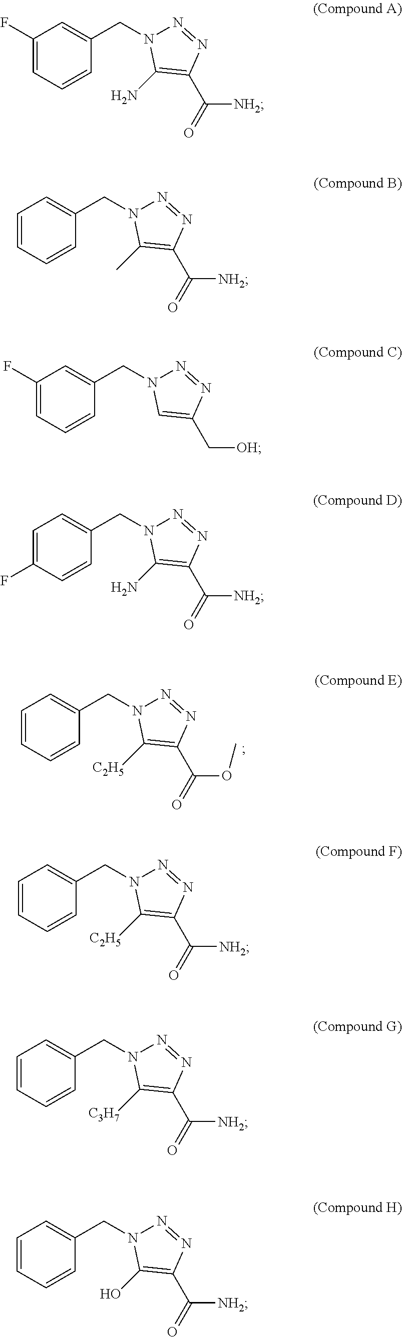

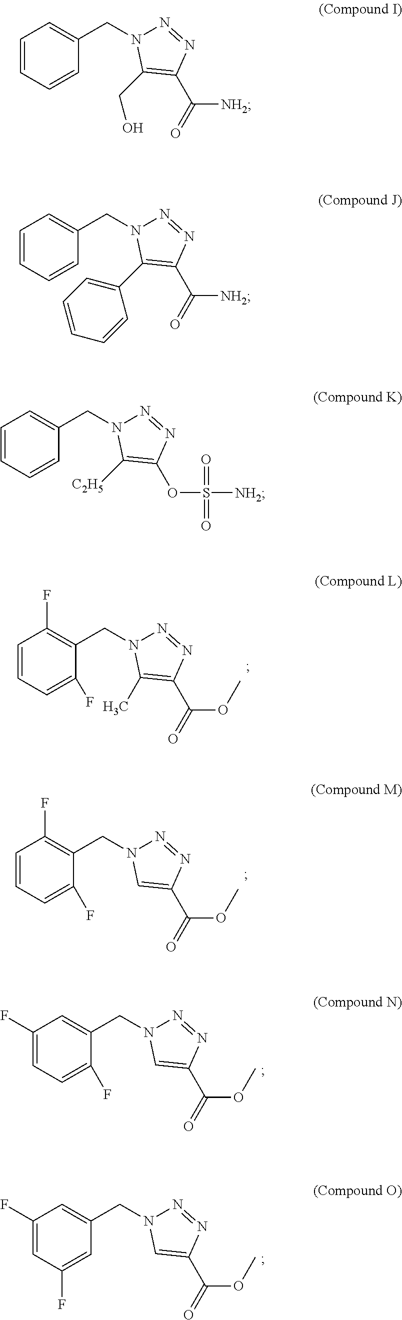

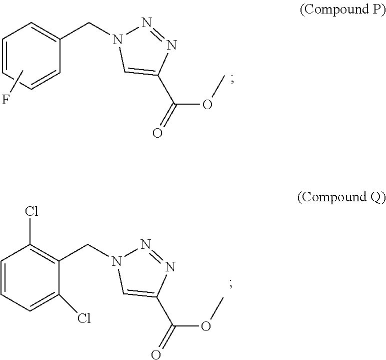

or a salt, solvate, or stereoisomer thereof.

Therefore, in accordance with an embodiment, the present invention provides the use of a composition comprising one or more one or more Naᵥ1.1 channel blockers to inhibit mechanical nociceptors on the myelinated neurons of a subject suffering from a neurological disorder, comprising administering to the subject, an effective amount of a composition comprising one or more Naᵥ1.1 channel blockers and a pharmaceutically acceptable carrier.

As used herein, the term “myelinated neurons” refers to those nerve fibers which are myelinated and have nociceptors. These nerve fibers are also referred to as A myelinated fibers or “AM” fibers or, in some embodiments, refer to Aδ pain fibers.

It will be understood, by those of skill in the art, that the axons associated with nociceptors, conduct relatively slowly, being only lightly myelinated or, more commonly, unmyelinated. Accordingly, axons conveying information about pain fall into either the Aδ group of myelinated axons, which conduct at about 20 m/s, as refered to in the present invention as AM fibers, or into the C fiber group of unmyelinated axons, which conduct at velocities generally less than 2 m/s. Thus, even though the conduction of all nociceptive information is relatively slow, there are fast and slow pain pathways.

In general, the faster-conducting Aδ nociceptors respond either to dangerously intense mechanical or to mechanothermal stimuli, and have receptive fields that consist of clusters of sensitive spots. Other unmyelinated nociceptors tend to respond to thermal, mechanical, and chemical stimuli, and are therefore said to be polymodal. In short, there are three major classes of nociceptors in the skin: Aδ mechanosensitive nociceptors, Aδ mechanothermal nociceptors, and polymodal nociceptors, the latter being specifically associated with C fibers. The receptive fields of all pain-sensitive neurons are relatively large, particularly at the level of the thalamus and cortex, presumably because the detection of pain is more important than its precise localization.

In accordance with an embodiment, the present invention provides the use of a composition comprising one or more one or more Naᵥ1.1 channel blockers to inhibit mechanical pain in a subject suffering from a neurological disorder, comprising administering to the subject, an effective amount of a composition comprising one or more Naᵥ1.1 channel blockers and a pharmaceutically acceptable carrier.

As used herein, the term “mechanical pain” can include pain due to mechanical or to mechanothermal stimuli.

In accordance with an embodiment, the present invention provides the use of a composition comprising one or more one or more Naᵥ1.1 channel blockers to inhibit allodynic pain in a subject suffering from a neurological disorder, comprising administering to the subject, an effective amount of a composition comprising one or more Naᵥ1.1 channel blockers and a pharmaceutically acceptable carrier.

As used herein, the term “allodynic pain” means a painful sensation caused by innocuous mechanical stimuli like light touch. Unlike inflammatory hyperalgesia that has a protective role, allodynia has no obvious biological utility. Allodynia is associated with nerve damage in conditions such as diabetes and fibromyalgia.

In accordance with an embodiment, the present invention provides the use of a composition comprising one or more one or more Naᵥ1.1 channel blockers to inhibit non-inflammatory pain in a subject suffering from a neurological disorder, comprising administering to the subject, an effective amount of a composition comprising one or more Naᵥ1.1 channel blockers and a pharmaceutically acceptable carrier.

As used herein, the term “inhibit non-inflammatory pain” means a painful sensation caused by an etiology other than inflammation of the tissue or tissue damage resulting from inflammation and inflammatory processes.

In accordance with an embodiment, the present invention provides the use of a composition comprising one or more one or more Naᵥ1.1 channel blockers to inhibit splanchnic colonic afferent neurons of a subject suffering from Irritable Bowel Syndrome (IBS), comprising administering to the subject, an effective amount of a composition comprising one or more Naᵥ1.1 channel blockers and a pharmaceutically acceptable carrier.

In accordance with an embodiment, the present invention provides the use of a composition comprising one or more one or more Naᵥ1.1 channel blockers to treat IBS in a subject suffering from IBS, or pain associated with IBS, comprising administering to the subject, an effective amount of a composition comprising one or more Naᵥ1.1 channel blockers and a pharmaceutically acceptable carrier.

The term, “peptide,” as used herein, includes a sequence of from four to 100 amino acid residues in length, preferably about 10 to 80 residues in length, more preferably, 15 to 65 residues in length, and in which the a-carboxyl group of one amino acid is joined by an amide bond to the main chain (a- or (3-) amino group of the adjacent amino acid. The peptides provided herein for use in the described and claimed methods and compositions can also be cyclic.

The precise effective amount for a human subject will depend upon the severity of the subject's disease state, general health, age, weight, gender, diet, time and frequency of administration, drug combination(s), reaction sensitivities, and tolerance or response to therapy. A routine experimentation can determine this amount and is within the judgment of the medical professional. Compositions may be administered individually to a patient, or they may be administered in combination with other drugs, hormones, agents, and the like.

Routes of administration of the inventive peptides and the one or more Naᵥ1.1 channel blockers include, but are not limited to, subcutaneously, intravenously, intraperitioneal, intracranial, intradermal, intramuscular, intraocular, intrathecal, intracerebrally, intranasally, infusion, via i.v. drip, patch and implant (e.g., pump).

In one or more embodiments, the present invention provides pharmaceutical compositions comprising one or more of the inventive peptides or one or more Naᵥ1.1 channel blockers and a pharmaceutically acceptable carrier. In other aspects, the pharmaceutical compositions also include one or more additional biologically active agents.

With respect to peptide compositions described herein, the carrier can be any of those conventionally used, and is limited only by physico-chemical considerations, such as solubility and lack of reactivity with the active compound(s), and by the route of administration. The carriers described herein, for example, vehicles, adjuvants, excipients, and diluents, are well-known to those skilled in the art and are readily available to the public. It is preferred that the carrier be one which is chemically inert to the active agent(s), and one which has little or no detrimental side effects or toxicity under the conditions of use. Examples of the carriers include soluble carriers such as known buffers which can be physiologically acceptable (e.g., phosphate buffer) as well as solid compositions such as solid-state carriers or latex beads.

The carriers or diluents used herein may be solid carriers or diluents for solid formulations, liquid carriers or diluents for liquid formulations, or mixtures thereof.

Solid carriers or diluents include, but are not limited to, gums, starches (e.g., corn starch, pregelatinized starch), sugars (e.g., lactose, mannitol, sucrose, dextrose), cellulosic materials (e.g., microcrystalline cellulose), acrylates (e.g., polymethylacrylate), calcium carbonate, magnesium oxide, talc, or mixtures thereof.

For liquid formulations, pharmaceutically acceptable carriers may be, for example, aqueous or non-aqueous solutions, or suspensions. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media.

Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Formulations suitable for parenteral administration include, for example, aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives.

In addition, in an embodiment, the compositions comprising the inventive peptides or derivatives thereof, or the one or more Naᵥ1.1 channel blockers, may further comprise binders (e.g., acacia, cornstarch, gelatin, carbomer, ethyl cellulose, guar gum, hydroxypropyl cellulose, hydroxypropyl methyl cellulose, povidone), disintegrating agents (e.g., cornstarch, potato starch, alginic acid, silicon dioxide, croscarmelose sodium, crospovidone, guar gum, sodium starch glycolate), buffers (e.g., Tris-HCl., acetate, phosphate) of various pH and ionic strength, additives such as albumin or gelatin to prevent absorption to surfaces, detergents (e.g., Tween 20, Tween 80, Pluronic F68, bile acid salts), protease inhibitors, surfactants (e.g. sodium lauryl sulfate), permeation enhancers, solubilizing agents (e.g., cremophor, glycerol, polyethylene glycerol, benzlkonium chloride, benzyl benzoate, cyclodextrins, sorbitan esters, stearic acids), anti-oxidants (e.g., ascorbic acid, sodium metabisulfite, butylated hydroxyanisole), stabilizers (e.g., hydroxypropyl cellulose, hyroxypropylmethyl cellulose), viscosity increasing agents (e.g., carbomer, colloidal silicon dioxide, ethyl cellulose, guar gum), sweetners (e.g., aspartame, citric acid), preservatives (e.g., thimerosal, benzyl alcohol, parabens), lubricants (e.g., stearic acid, magnesium stearate, polyethylene glycol, sodium lauryl sulfate), flow-aids (e.g., colloidal silicon dioxide), plasticizers (e.g., diethyl phthalate, triethyl citrate), emulsifiers (e.g., carbomer, hydroxypropyl cellulose, sodium lauryl sulfate), polymer coatings (e.g., poloxamers or poloxamines), coating and film forming agents (e.g., ethyl cellulose, acrylates, polymethacrylates), and/or adjuvants.

The choice of carrier will be determined, in part, by the particular peptide or the one or more Naᵥ1.1 channel blocker containing compositions, as well as by the particular method used to administer the composition. Accordingly, there are a variety of suitable formulations of the pharmaceutical compositions of the invention. More than one route can be used to administer the compositions of the present invention, and in certain instances, a particular route can provide a more immediate and more effective response than another route.

Injectable formulations are in accordance with the invention. The requirements for effective pharmaceutical carriers for injectable compositions are well-known to those of ordinary skill in the art (see, e.g., Pharmaceutics and Pharmacy Practice, J. B. Lippincott Company, Philadelphia, Pa., Banker and Chalmers, eds., pages 238-250 (1982), and ASHP Handbook on Injectable Drugs, Trissel, 15th ed., pages 622-630 (2009)).

As used herein the term “therapeutically active agent” or “biologically active agent” means an agent useful for the treatment or modulation of a disease or condition in a subject suffering therefrom. Examples of therapeutically active agents can include any drugs, peptides, siRNAs, and conjugates, known in the art for treatment of disease indications.

The biologically active agent may vary widely with the intended purpose for the composition. The term active is art-recognized and refers to any moiety that is a biologically, physiologically, or pharmacologically active substance that acts locally or systemically in a subject. Examples of biologically active agents, that may be referred to as “drugs”, are described in well-known literature references such as the Merck Index, the Physicians' Desk Reference, and The Pharmacological Basis of Therapeutics, and they include, without limitation, medicaments; vitamins; mineral supplements; substances used for the treatment, prevention, diagnosis, cure or mitigation of a disease or illness; substances which affect the structure or function of the body; or pro-drugs, which become biologically active or more active after they have been placed in a physiological environment.

Further examples of biologically active agents include, without limitation, enzymes, receptor antagonists or agonists, hormones and antibodies. Specific examples of useful biologically active agents include, for example, autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, a-blocker sympatholytics, sympatholytics, sympathomimetics, and adrenergic agonist sympathomimetics intravenous anesthetics, barbiturate intravenous anesthetics, benzodiazepine intravenous anesthetics, and opiate agonist intravenous anesthetics skeletal muscle relaxants, neuromuscular blocker skeletal muscle relaxants, and reverse neuromuscular blocker skeletal muscle relaxants; neurological agents, such as anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, such as antidepressants, heterocyclic antidepressants, monoamine oxidase inhibitors, selective serotonin re-uptake inhibitors, tricyclic antidepressants, antimanics, anti-psychotics, phenothiazine antipsychotics, anxiolytics, sedatives, and hypnotics, barbiturate sedatives and hypnotics, benzodiazepine anxiolytics, sedatives, and hypnotics, and psychostimulants.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect.

As used herein, the term “treat,” as well as words stemming therefrom, includes diagnostic and preventative as well as disorder remitative treatment.

Neurological disorders which involve, either directly or indirectly, Naᵥ1.1 channel modulating activity may be studied and/or treated using the peptides and pharmaceutical compositions comprising the inventive peptides or the one or more Naᵥ1.1 channel blockers. Known examples of diseases associated with the Naᵥ1.1 channel include: febrile epilepsy, GEFS+, Dravet syndrome (also known as severe myclonic epilepsy of infancy or SMEI), borderline SMEI (SMEB), West syndrome (also known as infantile spasms), Doose syndrome (also known as myoclonic astatic epilepsy), intractable childhood epilepsy with generalized tonic-clonic seizures (ICEGTC), Panayiotopoulos syndrome, familial autism, Rasmussens's encephalitis and Lennox-Gastaut syndrome. Based on the findings described herein, other examples of such diseases include, but are not limited to, Alzheimer's, migraine, including FHM3, and the treatment of acute and/or chronic pain associated with mechanosensitive neuronal fibers in disorders including, for example, Irritable Bowel Syndrome, static, mechanical or dynamic allodynias associated with neuropathies, complex regional pain syndrome, postherpetic neuralgia, fibromyalgia, spinal cord injury, menstrual cramps, other uterine pain and related diseases.

In some embodiments, the inventive peptides and the one or more Naᵥ1.1 channel blocker compositions can include imaging agents covalently linked to the peptides and compositions.

In accordance with an embodiment, the present invention provides a composition comprising one or more polypeptides having Naᵥ1.1 channel modulating activity described herein, and at least one or more imaging agents.

In accordance with another embodiment, the present invention provides a composition comprising one or more Naᵥ1.1 channel blockers, and at least one or more imaging agents.

In some embodiments, the imaging agent is a fluorescent dye. The dye may be an emitter in the visible or near-infrared (NIR) spectrum. Known dyes useful in the present invention include carbocyanine, indocarbocyanine, oxacarbocyanine, thuicarbocyanine and merocyanine, polymethine, coumarine, rhodamine, xanthene, fluorescein, borondipyrromethane (BODIPY), Cy5, Cy5.5, Cy7, VivoTag-680, VivoTag-S680, VivoTag-S750, AlexaFluor488, AlexaFluor660, AlexaFluor680, AlexaFluor700, AlexaFluor750, AlexaFluor790, Dy677, Dy676, Dy682, Dy752, Dy780, DyLight547, Dylight647, HiLyte Fluor 647, HiLyte Fluor 680, HiLyte Fluor 750, IRDye 800CW, IRDye 800RS, IRDye 700DX, ADS780WS, ADS830WS, and ADS832WS.

Organic dyes which are active in the NIR region are known in biomedical applications. However, there are only a few NIR dyes that are readily available due to the limitations of conventional dyes, such as poor hydrophilicity and photostability, low quantum yield, insufficient stability and low detection sensitivity in biological system, etc. Significant progress has been made on the recent development of NIR dyes (including cyanine dyes, squaraine, phthalocyanines, porphyrin derivatives and BODIPY (borondipyrromethane) analogues) with much improved chemical and photostability, high fluorescence intensity and long fluorescent life. Examples of NIR dyes include cyanine dyes (also called as polymethine cyanine dyes) are small organic molecules with two aromatic nitrogen-containing heterocycles linked by a polymethine bridge and include Cy5, Cy5.5, Cy7 and their derivatives. Squaraines (often called Squarylium dyes) consist of an oxocyclobutenolate core with aromatic or heterocyclic components at both ends of the molecules, an example is KSQ-4-H. Phthalocyanines, are two-dimensional 18π-electron aromatic porphyrin derivatives, consisting of four bridged pyrrole subunits linked together through nitrogen atoms. BODIPY (borondipyrromethane) dyes have a general structure of 4,4′-difluoro-4-bora-3a, 4a-diaza-s-indacene) and sharp fluorescence with high quantum yield and excellent thermal and photochemical stability.

Other imaging agents which can be attached to the inventive peptides or one or more Naᵥ1.1 channel blockers and compositions of the present invention include PET and SPECT imaging agents. The most widely used agents include branched chelating agents such as di-ethylene tri-amine penta-acetic acid (DTPA), 1,4,7,10-tetra-azacyclododecane-1,4,7,10-tetraacetic acid (DOTA) and their analogs. Chelating agents, such as di-amine dithiols, activated mercaptoacetyl-glycyl-glycyl-gylcine (MAG3), and hydrazidonicotinamide (HYNIC), are able to chelate metals like ^{99m}Tc and ¹⁸⁶Re. Instead of using chelating agents, a prosthetic group such as N-succinimidyl-4-¹⁸F-fluorobenzoate (¹⁸F-SFB) is necessary for labeling peptides with ¹⁸F. In accordance with an embodiment, the chelating agent is DOTA.

In accordance with an embodiment, the present invention provides the inventive peptides or one or more Naᵥ1.1 channel blockers attached to a metal isotope suitable for imaging. Examples of isotopes useful in the present invention include Tc-94m, Tc-99m, In-111, Ga-67, Ga-68, Y-86, Y-90, Lu-177, Re-186, Re-188, Cu-64, Cu-67, Co-55, Co-57, Sc-47, Ac-225, Bi-213, Bi-212, Pb-212, Sm-153, Ho-166, or Dy-i66.

In accordance with an embodiment, the present invention provides peptides or one or more Naᵥ1.1 channel blockers and compositions wherein the imaging agent portion comprises ¹¹¹In labeled DOTA which is known to be suitable for use in SPECT imaging.

In accordance with another embodiment, the present invention provides a peptides or one or more Naᵥ1.1 channel blockers and compositions wherein the imaging agent comprises Gd³⁺ labeled DOTA which is known to be suitable for use in MR imaging. It is understood by those of ordinary skill in the art that other suitable radioisotopes can be substituted for ¹¹¹In and Gd³⁺ disclosed herein.

In accordance with an embodiment, the present invention provides the use of compositions comprising one or more polypeptides having Naᵥ1.1 channel modulating activity described herein, or more Naᵥ1.1 channel blockers covalently linked to at least one or more imaging agents for diagnosis of neurological disorders which involve, either directly or indirectly, Naᵥ1.1 channel modulating activity in a subject in need thereof, comprising administering to the subject an effective amount of compositions comprising one or more polypeptides having Naᵥ1.1 channel modulating activity, or more Naᵥ1.1 channel blockers covalently linked to at least one or more imaging agents and a pharmaceutically acceptable carrier.

Examples of diseases where such imaging agents can be used include, but are not limited to: febrile epilepsy, GEFS+, Dravet syndrome, borderline SMEI (SMEB), West syndrome, Doose syndrome, intractable childhood epilepsy with generalized tonic-clonic seizures (ICEGTC), Panayiotopoulos syndrome, familial autism, Rasmussens's encephalitis, Lennox-Gastaut syndrome, migraine, including FHM3, acute and/or chronic pain associated with mechanosensitive neuronal fibers in disorders including, for example, Irritable Bowel Syndrome, static, mechanical or dynamic allodynias associated with neuropathies, complex regional pain syndrome, postherpetic neuralgia, fibromyalgia, spinal cord injury, and related diseases.

In accordance with an embodiment, the present invention provides one or more nucleic acid sequences encoding any of the polypeptides having Naᵥ1.1 channel modulating activity or derivatives, homologues, analogues or mimetics thereof disclosed herein.

By “nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acids of the invention are recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

In accordance with an embodiment, the present invention provides one or more non-naturally occurring cDNA sequences encoding any of the polypeptides having Naᵥ1.1 channel modulating activity or derivatives, homologues, analogues or mimetics thereof disclosed herein.

The nucleic acids can be constructed based on chemical synthesis and/or enzymatic ligation reactions using procedures known in the art. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acids can be constructed based on chemical synthesis and/or enzymatic ligation reactions using procedures known in the art. See, for example, Sambrook et al. (eds.), Molecular Cloning, A Laboratory Manual, 3rd Edition, Cold Spring Harbor Laboratory Press, New York (2001) and Ausubel et al., Current Protocols in Molecular Biology, Greene Publishing Associates and John Wiley & Sons, NY (2007). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

In accordance with another embodiment, the present invention provides a vector comprising one or more nucleic acid sequences encoding any of the polypeptides having Naᵥ1.1 channel modulating activity or derivatives, homologues, analogues or mimetics thereof disclosed herein.

The nucleic acids of the invention can be incorporated into a recombinant expression vector. In this regard, the invention provides recombinant expression vectors comprising any of the nucleic acids of the invention. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors of the invention are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring internucleotide linkages, or both types of linkages. Preferably, the non-naturally occurring or altered nucleotides or internucleotide linkages do not hinder the transcription or replication of the vector.

The recombinant expression vectors of the invention can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell, such as Xenopus oocytes. Replication systems can be derived, e.g., from ColEl, 2 μ plasmid, λ, SV40, bovine papilloma virus, and the like.

Desirably, the recombinant expression vector comprises regulatory sequences, such as transcription and translation initiation and termination codons, which are specific to the type of host (e.g., bacterium, fungus, plant, or animal) into which the vector is to be introduced, as appropriate and taking into consideration whether the vector is DNA or RNA based.

The recombinant expression vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, LacZ, green fluorescent protein (GFP), luciferase, neomycin/G418 resistance genes, hygromycin resistance genes, histidinol resistance genes, tetracycline resistance genes, and ampicillin resistance genes.

The heterologous nucleic acid can be a nucleic acid not normally found in the target cell, or it can be an extra copy or copies of a nucleic acid normally found in the target cell. The terms “exogenous” and “heterologous” are used herein interchangeably.

The invention further provides a host cell comprising any of the recombinant expression vectors described herein. As used herein, the term “host cell” refers to any type of cell that can contain the inventive recombinant expression vector. The host cell can be an animal cell. Preferably, in an embodiment, the host cell is a mammalian cell. The host cell can be a cultured cell or a primary cell, i.e., isolated directly from an organism, e.g., a human. The host cell can be an adherent cell or a suspended cell, i.e., a cell that grows in suspension. Most preferably, the host cell is a human cell. The host cell can be of any cell type, can originate from any type of tissue, and can be of any developmental stage. Most preferably the host cells can include, for instance, muscle, lung, and brain cells, and the like.

The host referred to in the inventive methods can be any host. Preferably, the host is a mammal.

As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

Also provided by the invention is a population of cells comprising at least one host cell described herein. The population of cells can be a heterogeneous population comprising the host cell comprising any of the recombinant expression vectors described, in addition to at least one other cell, e.g., a host cell (e.g., a nerve cell), which does not comprise any of the recombinant expression vectors, or a cell other than a nerve cell, e.g., a skin cell, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly of host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

The invention further encompasses screening methods to identify small molecules, or derivatives and analogs of the inventive peptides which have Naᵥ1.1 channel modulating activity. Such methods would include use of a preparation of a cell or population of cells which comprise the Naᵥ1.1 channel and contacting the cell or population of cells with a test compound and determining the channel activity in the presence of the test compound. This could be followed or preceded by contacting the cell or population of cells with one or more of the inventive peptides and determining the channel activity in the presence of the inventive peptides. The cell or population of cells could then be contacted with the test compound and/or with the inventive peptides in the presence of a Naᵥ1.1 channel blocker, such as ICA-121431, and the channel activity would then be determined.

If the test compound selectively activates the Naᵥ1.1 channel in an amount equal to, or greater than the amount of activation of the inventive peptides, and where the activation of the Naᵥ1.1 channel by the test compound is inhibited when in the presence of a Naᵥ1.1 channel blocker, a determination is made that the test compound is a selective Naᵥ1.1 channel activator.

If the test compound selectively activates the Naᵥ1.1 channel in an amount less than the amount of activation of the inventive peptides, and where the activation of the Naᵥ1.1 channel by the inventive peptides is inhibited when in the presence of the target compound at an amount equal to or greater that a Naᵥ1.1 channel blocker, a determination is made that the test compound is a selective Naᵥ1.1 channel blocker.

The cell or population of cells used in the screening methods disclosed herein can be any cell which comprises one or more the Naᵥ1.1 channels. For example, the cells can be neuronal or non-neuronal cells which have been transfected with a vector comprising a nucleic acid which encodes the Naᵥ1.1 channel and which is expressed by the cells. In some embodiments, the cells can be Xenopus laevis oocytes, for example. In some embodiments the cell or population of cells can be cultured neuronal cells which comprise the Naᵥ1.1 channel. Any known neuronal cell culture either as an immortalized cell line, or primary cultured neurons which comprise the Naᵥ1.1 channel, can be used. In some other embodiments, ex vivo preparation of whole nerves can be used to screen compounds. For example, cutaneous nerves of the limbs of mice, such as the saphenous nerve, can be used in ex vivo preparations known to those of ordinary skill, and those nerves can be exposed to the test compounds and inventive peptides and the neuronal activity can be determined.

In some embodiments, nerves of the gut from mammals can be removed and in vitro recordings of action potential discharges can be made. In some embodiments, the nerves used are splanchnic colonic afferent nerves.

In accordance with one or more embodiments, nerve preparations from normal healthy mice and mice with chronic visceral mechanical hypersensitivity (CVH), which is a mouse model for IBS can be used to screen test compounds and the activities of the test and control compounds on normal and CVH neurons can be compared. For example, colonic afferent nerves from CVH mice and normal controls can be exposed to a test compound and then mechanosensory responses are measured. If a test compound lessens the mechanosensory responses of the CVH mice compared to normal and in comparison to control compounds, then the test compound is determined to be a Naᵥ1.1 channel blocker and may be useful in the treatment of pain associated with IBS.

In accordance with one or more embodiments, the measurement of the activity of the Naᵥ1.1 channel in any of the above methods can be performed using known electrophysiological methods in the art. Examples of such methods include, but are not limited to, (automated) patch clamp methods, two-electrode voltage-clamp recording techniques, cut-open oocyte Vaseline gap technique, and other methods.

In some embodiments, the measurement of the activity of the Naᵥ1.1 channel in any of the above methods can be performed using known imaging methods in the art. For example, Naᵥ1.1 channels in cells can be measured using fluorescence or luminescence detection methods such as fluorescence imaging plate reader (FLIPR) technology in combination with Nadi channel modulators such as veratridine.

### EXAMPLES

Venom Collection and Screening.

Venoms from spiders, scorpions and centipedes were collected by mild electrical stimulation, then dried and kept frozen until used. 109 venoms were tested by ratiometric calcium imaging using a standard inverted microscope setup. Responses were digitized and analyzed using MetaMorph software (Molecular Devices). Venom-evoked responses that were stimulus-locked, visually detectable above background, and restricted to neurons (i.e. did not cause calcium entry into glia or fibroblasts). Pharmacological analysis was used to narrow down potential targets and crude venoms or purified fractions were subsequently tested on candidate cloned channels. Candidates were taken forward based on robustness of the response and evidence for selectivity at novel targets.

Hm1a/b Isolation.

Venom from H. maculata (1 mg dried) was fractionated on a C₁₈ reversed-phase (RP) high-performance liquid chromatography (HPLC) column (Jupiter 250×4.6 mm, 5 mm; Phenomenex, Torrance, Calif.) on a Shimadzu (Shimadzu, Rydalmere, NSW, Australia) Prominence HPLC system. The following linear gradients of solvent B (90% acetonitrile, 0.1% formic acid in water) in solvent A (0.1% formic acid in water) were used at a flow rate of 1 ml/min: 5% B for 5 min, then 5-20% B for 5 min followed by 20-40% B over 40 min. Absorbance was determined at 214 nm and 280 nm and collected fractions were lyophilized before storage at −20° C.

Mass Spectrometry.

Peptide masses were determined by matrix-assisted laser desorption/ionization (MALDI) time of flight (TOF) mass spectrometry (MS) using a 4700 Proteomics Bioanalyzer model (Applied Biosystems, Carlsbad, Calif.). Peptides were dissolved in water and mixed 1:1 (v/v) with a-cyano-4-hydroxycinnamic acid matrix (7 mg/ml in 50% acetonitrile, 5% formic acid) and mass spectra acquired in positive reflector mode. All reported masses are for the monoisotopic M+H⁺ ions.

Edman Sequencing.

N-terminal sequencing was performed by the Australian Proteome Analysis Facility (Sydney, NSW, Australia). In brief, Hm1a (600 pmol) and Hm1b (250 pmol) were reconstituted and reduced using DTT (25 mM) and left to incubate at 56° C. for 0.5 h. The samples were then alkylated using iodoacetamide (55 mM) at room temperature for 0.5 h and purified by RP-HPLC using a Zorbax 300SB-C18 column (3×150 mm). The target peaks of interest were identified, collected then reduced to minimal volume under vacuum. The entire sample was loaded onto a precycled, Biobrene-treated disc and was subjected to 37 (Hm1a) or 42 (Hm1b) cycles of Edman N-terminal sequencing, respectively. Automated Edman degradation was carried out using an Applied Biosystems 494 Procise Protein Sequencing System.

Sequence Determination.

Edman sequencing for Hm1a yielded ECRYLFGGCSSTSDCCKHLSCRSDWKYCAWDGTF (SEQ ID NO: 3) as the sequence, which has a calculated monoisotopic mass (for the M+H⁺ ion) of 3908.58 Da. This is 86.97 Da short of the monoisotopic mass of Hm1a of 3995.55 Da. Hence, we conclude that an ‘S’ (87 Da) is missing on the C-terminal end of Hm1a to give a complete sequence of ECRYLFGGCSSTSDCCKHLSCRSDWKYCAWDGTFS (SEQ ID NO: 1). The complete sequence has a calculated monoisotopic mass (for the M+H⁺ ion) of 3995.61 Da, which is only 0.06 Da different to the mass that was measured for the native Hm1a.

Edman sequencing for Hm1b yielded ECRYLFGGCKTTADCCKHLGCRTDLYYCAWDGT (SEQ ID NO: 4) as the sequence, which has a calculated monoisotopic mass (for the M+H⁺ ion) of 3745.6 Da. This is 147 Da short of the monoisotopic mass of Hm1a of 3892.60 Da. We therefore conclude that an amidated ‘F’ is missing on the C-terminal end of Hm1b to give a complete sequence of ECRYLFGGCKTTADCCKHLGCRTDLYYCAWDGTF-NH2 (SEQ ID NO: 2). C-terminal amidation was confirmed by digesting 4 ug of the native Hm1b with Carboxypeptidase Y for 20 minutes and measuring the mass difference between the intact and digested Hm1b. We found this difference to be 146 Da, corresponding to the final residue, Phe, with an amidated C-terminus. The complete sequence has a calculated monoisotopic mass (for the M+H⁺ ion) of 3892.64 Da, matching the native Hm1b.

To confirm that the C-terminus of Hm1b is amidated, we digested native Hm1b with Carboxypeptidase Y (CPY) and monitored the reaction by MALDI-TOF to identify the mass of the C-terminal residue as described previously⁵¹. 5 μL of 800 ng/μL native Hm1b in 100 mM ammonium acetate, pH 5.5, was incubated with 2 ng/μL CPY at 37° C. for 20 min. The reaction was monitored by removing 0.4 μL at 0, 1, 5, 10 and 20 min and spotting it on a MALDI plate with equal volume of 7 mg/mL a-cyano-4-hydroxycinnamic acid in 60% (v/v) acetonitrile, 5% formic acid (FA). Dried spots were washed with 10 μL 1% FA and allowed to dry before they were analyzed by MALDI-TOF-MS on a 4700 Proteomics Bioanalyser (Applied Biosciences, Foster City, Calif., USA), acquiring spectra in reflector positive mode.

Hm1a Synthesis.

Solvents for reversed-phase HPLC consisted of 0.05% TFA/H₂O (A) and 90% MeCN/0.043% TFA/H₂O (B). Analytical HPLC was performed on a Shimadzu LC20AT system using a Thermo Hypersil GOLD 2.1×100 mm C18 column heated at 40° C. with flow rate of 0.3 mL/min. A gradient of 10 to 55% B over 30 min was used, with detection at 214 nm. Preparative HPLC was performed on a Vydac 218TP1022 column running at a flow rate of 16 mL/min using a gradient of 10 to 50% B over 40 min. Mass spectrometry was performed on an API2000 (ABI Sciex) mass spectrometer in positive ion mode. All reagents were obtained commercially and were used without further purification.

Peptide Synthesis.

Hm1a was synthesized using regioselective disulfide-bond formation^{52,54}. The peptide was assembled on a 0.1 mmol scale using a Symphony (Protein Technologies Inc.) automated peptide synthesizer and a H-Ser(tBu)-2-C1Trt (loading 0.69 mmol/g) polystyrene resin. Couplings were performed in DMF using 5 equivalents of Fmoc-amino acid/HBTU/DIEA (1:1:1) relative to resin loading for 2×20 min. Fmoc deprotection was achieved using 30% piperidine/DMF (1×1.5 min, then 1×4 min). Non-cysteine amino acid side-chains were protected as Asp(OtBu), Arg(Pbf), Glu(OtBu), His(Trt), Lys(Boc), Ser(tBu), Thr(tBu), Trp(Boc) and Tyr(tBu). The cysteine side chains were protected as Cys2,Cys16(Meb), Cys9,Cys21(Dpm), and Cys15,Cys28(Trt). Cleavage from the resin was achieved by treatment with 10% AcOH/10% TFE/DCM at room temperature for 1 h. The product was precipitated and washed with n-hexane then lyophilised from 1,4-dioxane/MeCN/H₂O.

The first disulfide bond (Cys15-Cys28) was formed by dissolving the crude product in in HFIP (5 mL) and adding dropwise to a stirred solution of 12 (4 equiv) in 10% HFIP/DCM (20 mL) over 5 min. Stirring was continued for a further 5 min then the solution was poured into a solution of ascorbic acid/NaOAc in H2O. The aqueous phase was extracted with DCM, and the combined organic layers washed with water. Following removal of solvent under reduced pressure, the product was lyophilised from 1,4-dioxane/MeCN/H20. ESI-MS (m/z): calc. (avg) 2159.4[M+3H]³⁺, found 2159.7.

The remaining side chain protecting groups [except Cys(Meb)] were removed by treatment with 95% TFA/2.5% TIPS/2.5% H₂O at room temperature for 2 h. After most of the cleavage solution was evaporated under a stream of N₂, the product was precipitated and washed with cold Et₂O and lyophilised from 50% MeCN/0.1% TFA/H₂to give Cys2,Cys16(Meb), Cys9,Cys21(SH), Cys15-Cys28(SS) Hm1a (280 mg). ESI-MS (m/z): calc. (avg) 1404.3[M+3H]³⁺, found 1404.1.

The second disulfide bond (Cys9-Cys21) was formed by dissolving the crude product from the previous step in 30% DMSO/0.1M HCl (0.5 mg/mL) and stirring at room temperature for 24 h. Cys2,16(Meb), Cys9-Cys21(SS), Cys15-Cys28(SS) Hm1a was then isolated by preparative HPLC (30 mg). ESI-MS (m/z): calc. (avg) 1403.6[M+3H]³⁺, found 1403.3.

Formation of the third disulfide bond (Cys2-Cys16) was then achieved by first removing the Cys(Meb) groups by treatment with HF/p-cresol (9:1) at 0° C. for 1 h. The product was precipitated and washed with cold Et₂O and lyophilised from 50% MeCN/0.1% TFA/H₂O yielding Cys2,16(SH), Cys9-Cys21(SS), Cys15-Cys28(SS) Hm1a (24 mg). ESI-MS (m/z): calc. (avg) 1334.1[M+3H]³⁺, found 1333.7. Oxidation of the liberated thiols was performed using 30% DMSO/0.1M HCl as described for the second disulfide bond to yield fully oxidised Hm1a (3 mg) that was indistinguishable by analytical HPLC from an authentic sample. ESI-MS (m/z): calc. (avg) 1333.5[M+3H]³⁺, found 1333.1.

Abbreviations: DCM, dichloromethane; DIEA, N,N-diisopropylethylamine; DMF, N,N-dimethylformamide; HBTU, 2-(1H-benzotriazol-1-yl)-1,1,3,3-tetramethyluronium hexafluorophosphate; HFIP, 1,1,1,3,3,3-hexafluoropropan-2-ol; MeCN, acetonitrile; TFA, trifluoroacetic acid; TIPS, triisopropylsilane.

Naᵥ and Kᵥ Channel Constructs.

Human (h)Naᵥ1.4, hNaᵥ1.5, and rat (r)Kᵥ2.1 were a gift from Peter Ruben (Simon Fraser University, Canada), Chris Ahern (University of Iowa, USA), and Kenton J Swartz (NINDS, NIH, USA), respectively. hNaᵥ1.1-1.3, hNaᵥ1.6-1.8 were obtained from Origene Technologies, Inc. (MD, USA). Accession numbers are NM_001165963.1 (hNaᵥ1.1), NM_021007.2 (hNaᵥ1.2), NM_006922.3 (hNaᵥ1.3), NM_000334 (hNaᵥ1.4), NM_198056 (hNaᵥ1.5), NM_014191.2 (hNaᵥ1.6), NM_002977.2 (hNaᵥ1.7), and NM_006514.3 (hNaᵥ1.8). Channel chimeras were generated using sequential PCR with rNaᵥ1.4 (gift from Baron Chanda, University of Wisconsin, USA), Kᵥ2.1Δ7^{55,56}, hNaᵥ1.1, and hNaᵥ1.9²⁴ (Origene Technologies: NM_014139.2) as templates. Mouse Kᵥ4.1 was obtained from AddGene and originated in the laboratory of Dr. Lawrence Salkoff. The Kᵥ2.1Δ7 construct contains seven point mutations in the outer vestibule that render the channel sensitive to agitoxin-2, a pore-blocking scorpion toxin⁵⁷. cRNA of all constructs was synthesized using T3 or T7 polymerase (mMessage mMachine kit, Life technologies, USA) after linearizing the fully-sequenced DNA with appropriate restriction enzymes.

Electrophysiology.

Xenopus Oocytes.

Channels and chimeras were expressed in Xenopus laevis oocytes (animals acquired from Xenopus one®, USA) that were incubated at 17° C. in Barth's medium (88 mM NaCl, 1 mM KCl, 0.33 mM Ca(NO₃)₂, 0.41 mM CaCl₂, 0.82 mM MgSO₄, 2.4 mM NaHCO₃, 5 mM HEPES, and 0.1 mg/mL gentamycin; pH 7.6 with NaOH) for 1-4 days after cRNA injection, and then were studied using two-electrode voltage-clamp recording techniques (OC-725C; Warner Instruments or GeneClamp 500B; Axon Instruments) with a 150-μl recording chamber or a small volume (<20 μl) Oocyte Perfusion Chamber (AutoMate Scientific). Data were filtered at 4 kHz and digitized at 20 kHz using pClamp 10 software (Molecular Devices, USA). Microelectrode resistances were 0.5-1 MΩ when filled with 3 M KCl. For Kᵥ channel experiments, the external recording solution contained (in mM): 50 KCl, 50 NaCl, 5 HEPES, 1 MgCl2 and 0.3 CaCl₂, pH 7.6 with NaOH. For Naᵥ channel experiments, the external recording solution contained (in mM): 100 NaCl, 5 HEPES, 1 MgCl₂ and 1.8 CaCl₂, pH 7.6 with NaOH. All experiments were performed at room temperature (˜22° C.) and toxin samples were diluted in recording solution with 0.1% BSA. Leak and background conductance, identified by blocking the channel with agitoxin-2 or TTX, were subtracted for Kᵥ or Naᵥ channel currents, respectively. Voltage-activation relationships were obtained by measuring tail currents for Kᵥ channels, or by monitoring steady-state currents and calculating conductance for Naᵥ channels. Occupancy of closed or resting channels by toxins was examined using negative holding voltages where open probability was low, and the fraction of unbound channels was estimated using depolarizations that are too weak to open toxin-bound channels. After addition of toxin to the recording chamber, equilibration between toxin and channel was monitored using weak depolarizations elicited at 5-10 s intervals. For all channels, voltage-activation relationships were recorded in the absence and presence of toxin. Off-line data analysis was performed using Clampfit 10 (Molecular Devices, USA) and Origin 7.5 (Originlab, USA).

Multiple protocols were used to probe the biophysical characteristics of the Naᵥ channels and chimeras studied. To determine conductance-voltage and steady-state inactivation relationships, oocytes expressing Naᵥ channels were held at −90 mV and depolarized in 5 mV steps from −90 mV to 5 mV for 50 ms, immediately followed by a step to −15 mV to elicit the maximum available current and after 50 ms, returned to the −90 mV holding potential. Peak current generated during the incremental portion of the protocol was used to calculate the conductance-voltage relationship while the peak current during the −15 mV step as a function of the earlier voltage step was used to determine the steady-state inactivation relationship. The time constant of fast inactivation was determined by fitting single exponential curves to the −15 mV step of the aforementioned protocol. Boltzmann curves were fitted in Clampfit 10 (Molecular Devices, USA) and statistics calculated with Excel or the R statistical package (Student's t-test).

Cultured Neurons.

Whole cell patch clamp of cultured mouse TG neurons was performed as described⁵⁸. Buffer solution contained (in mM) 150 NaCl, 2.8 KCl, 1 MgSO₄, 10 HEPES, pH 7.4 (NaOH) and was perfused with or without toxins/drugs using a SmartSquirt Micro-Perfusion system (AutoMate). For colonic DRG, Whole-cell recordings were made from fluorescently labeled thoracolumbar (T10-L1) colonic DRG neurons 20-48 h after plating, using fire-polished glass electrodes with a resistance of 2-5 MΩ. All recordings were performed at room temperature (20-22° C.). Signals were amplified by using an Axopatch 200A amplifier, digitised with a Digidata 1322A and recorded using pCLAMP 9 software (Molecular Devices, Sunnyvale, Calif., USA). For all DRG neurons the holding potential was −70 mV. In current clamp mode a series of depolarizing pulses (500 ms, 10 pA step) were applied from holding potential (−70mV) and the rheobase (amount of current (pA) required for action potential generation) determined. The number of action potentials at 2× rheobase was also determined. Depolarizing pulses were tested in normal external bath solution and following the addition of Hm1a (100 nM). Control solutions and Hm1a were applied with a gravity driven multi-barrel perfusion system positioned within 1mm of the neuron under investigation. Intracellular solutions contained (mM): KCl, 135; MgCl₂, 2; MgATP, 2; EGTA-Na, 5; Hepes-Na, 10; adjusted to pH 7.4. Extracellular solutions contained (mM): NaCl, 140; KCl, 4; MgCl₂, 2; CaCl₂, 2; Hepes-Na, 10; glucose, 5; adjusted to pH 7.4.

Skin-Nerve Recordings.

To assess primary afferent activity in response to the Hm1a spider toxin, we utilized the ex vivo skin-nerve preparation, as previously described⁵⁹. Briefly, animals were lightly anesthetized via inhaled isoflurane and then killed via cervical dislocation. The hair on the lower extremities was shaved, and the hairy skin of the hindpaw was then quickly dissected along with its innervating saphenous nerve. The skin and nerve were then placed in a recording chamber filled with warmed (32° C.), oxygenated buffer consisting of (in mM): 123 NaCl, 3.5 KCl, 2.0 CaCl₂, 1.7 NaH₂PO₄, 0.7 MgSO₄, 9.5 sodium gluconate, 5.5 glucose, 7.5 sucrose and 10 HEPES titrated to a pH of 7.45±0.05.

The nerve was then threaded into a mineral oil-filled chamber, teased apart atop an elevated mirror plate, and placed on an extracellular recording electrode. Single unit receptive fields were then identified via a mechanical search stimulus utilizing a blunt glass probe. Aδ afferents were identified based on a conduction velocity between 1.2 and 10 m/s, and were subtyped into A-mechanonociceptors (AM's) based on their slow adaptation to a mechanical stimulus⁶⁰.

After locating an AM fiber, its von Frey threshold was obtained by stimulating the receptive field with calibrated von Frey filaments to determine the threshold force for action potential generation. A metal moat (inner diameter: 4.7 mm) was then placed over the center of the receptive field to isolate it from the surrounding buffer. Buffer within the moat was then evacuated and replaced with a buffer containing either 1 μM Hm1a or vehicle (buffer). Receptive fields were incubated with toxin or buffer for 2-5 minutes. A custom-built, feedback-controlled mechanical stimulator was then placed within the moat and the receptive field was mechanically stimulated with a series of increasing forces (15 mN, 50 mN, 100 mN) for 10 seconds each. A rest period of 1 minute was given between stimulations to avoid sensitization/desensitization.

Data was digitized using a PowerLab A/D converter (AD Instruments, USA) and recorded using LabChart software and Spike Histogram extension (AD Instruments, USA). All skin-nerve data was recorded and analyzed with the experimenter blinded to whether toxin or vehicle was used. Recordings were only included in the final data set if the firing of the fiber was clearly distinguishable from both background noise and any other fibers firing during stimulation.

Gut-Nerve Recordings.

In vitro single-unit extracellular recordings of action potential discharge were made from splanchnic colonic afferents. Recordings were made from healthy or CVH mice using standard protocols⁶¹⁻⁶³. Baseline mechanosensitivity was determined in response to application of a 2 g von frey hair (vfh) probe to the afferent receptive field for 3 seconds. This process was repeated 3-4 times, separated each time by 10 seconds. Mechanosensitivity was then re-tested after the application of Hm1a (100 nM) or the Naᵥ1.1 blocker ICA-121432 (500 nM) or a combination of both ICA-121432 (500 nM) and Hm1a (100 nM). Data are presented as spikes/s and are expressed as mean±SEM.

Animal Use, Husbandry and Genotyping.

Mice were bred and housed in accordance with UCSF Institutional Animal Care Committee (IACUC) guidelines. 2-5 animals were housed together with constant access to food and water. Floxed SCN1a mice¹³ were generously provided by Dr. William Catterall (Dept. of Pharmacology, University of Washington). Floxed mice were bred to Peripherin Cre (Per-Cre) mice³⁷ to produce SCN1a^{F/F}×Per-Cre conditional knockout mice. Naᵥ1.1 floxed alleles were detected using primers previously described (Cheah) and Per-Cre expression was detected using the following primers to Cre recombinase: Cre_F: TAGCGTTCGAACGCACTGATTTCG (SEQ ID NO: 5); Cre_R: CGCCGTAAATCAATCGATGAGTTG (SEQ ID NO: 6).

Somatic behavioral experiments were approved by UCSF IACUC and were in accordance with the National Institutes of Health (NIH) Guide of the Care and Use of Laboratory Animals and the recommendation of the International Association for the Study of Pain. Animals used in skin-nerve recordings were naïve C57b1/6 male mice (n=10), aged 6-16. Mice were housed on a 14:10 light:dark cycle with ad libitum access to food and water in a climate-controlled room. All protocols were approved by the Institutional Animal Care and Use Committee at the Medical College of Wisconsin. Animals used in colonic afferent and colonic DRG neuron studies were male C57BL/6J mice. The Animal Ethics Committees of The University of Adelaide and the South Australian Health and Medical Research Institute (SAHMRI) approved experiments involving animals.

Sensory Neuron Culture and Calcium Imaging.

Trigeminal ganglia were dissected from newborn (P0-P3) Sprague-Dawley rats or C57BL/6 mice and cultured for >12 hours before calcium imaging or electrophysiological recording. Embryonic DRG cultures were generously provided by Jonah Chan⁶⁴. Embryonic cultures were maintained as described and calcium imaging experiments were performed 1-10d after primary cultures were established. Primary cells were plated onto cover slips coated with Poly-L-lysine (Sigma) and laminin (Invitrogen—10 μg/ml). Cells were loaded for calcium imaging with Fura-2-AM (Molecular Probes) for >1 hour. Buffer solution—(in mM) 150 NaCl, 2.8 KCl, 1 MgSO₄, 10 HEPES, pH 7.4 (NaOH)—was perfused with or without toxins/drugs using a SmartSquirt Micro-Perfusion system (AutoMate).

In situ Hybridization and Immunohistochemistry.

In situ hybridization (ISH) was performed using the ViewRNA ISH Tissue 2-plex or 1-plex Assay Kits (Affymetrix). Target mRNA signals appear as puncta in bright field or fluorescent microscopy. Eight to twelve week old mice were deeply anesthetized with pentobarbital then transcardially perfused with 10 ml of phosphate buffered saline (PBS) followed by 10 ml of 10% neutral buffered formalin (NBF). DRGs were dissected, post-fixed in 10% NBF at 4° C. O/N, cryoprotected in PBS with 30% w/v sucrose O/N at 4° C., then embedded in OCT Compound at −20° C. Tissue was sectioned at 12 μm, thaw-captured on Diamond White Glass slides (Globe Scientific), and stored at −20° C. until use. Slides were used within two weeks of processing to produce optimal signals.

ViewRNA ISH Tissue 2-plex assay was performed with frozen tissue modifications as indicated by manufacturer including the endogenous alkaline phosphatase inactivation by incubation in H₂O with 0.1M HCl and 300 mM NaCl. H&E counterstaining procedure was omitted. Images were acquired with a Leica DMRB microscope and DFC500 digital camera using Leica Application Suite v3.5.0 then further analyzed using ImageJ software.

To co-label neuronal subpopulations markers (NF200, IB4, CGRP, TH) and Naᵥ1.1 mRNA, ViewRNA ISH Tissue 1-plex Assay and immunohistochemistry were performed sequentially using a protocol modified from⁶⁵. ISH/IHC was not found to be compatible with all primary antibodies. Animals, tissue, and slides were prepared as described in the preceding paragraph. Frozen slides with tissue sections were warmed in a vacuum oven for 10 minutes at 60° C., fixed in PBS with 4% v/v formaldehyde for 10 minutes at RT then processed according to the manufacturer's protocol with frozen tissue modifications in a ThermoBrite Slide Processing System (Abbott Molecular). Washing steps were performed as indicated, in a deliberate and vigorous manner. Optimal protease and probe incubation times were determined to be 12 minutes and 2 hours, respectively. Following development in Fast Red Substrate, slides were rinsed briefly in PBS then immediately processed for immunohistochemistry. Slides were incubated for one hour in a blocking solution at room temperature (RT) consisting of PBS with 0.1% v/v Triton X-100 (Sigma) and 10% normal goat serum (NGS). Slides were then incubated in primary antibody solution (PBS with 0.1% Triton X-100 and 2.5% NGS) O/N at 4° C., vigorously agitated for 2 min in fresh PBS 3×, then incubated in secondary antibody solution (PBS with 0.1% v/v Triton X-100) for 2 hr at RT in the dark. Sections were then washed by vigorous agitation for 2 min in fresh PBS 3× prior to mounting with ProLong Gold antifade reagent with DAPI (Life Technologies) and coverslipping. Images were acquired with a Leica DMRB microscope and DFC500 digital camera using Leica Application Suite v3.5.0 then further analyzed using ImageJ software.

Affymetrix was commissioned to design a Type 1 probe set to mouse Naᵥ1.1 (Scn1a, NM_018733.2) and Type 6 probe sets to mouse TRPV1 (TrpV1, NM_001001445.2), mouse Naᵥ1.7 (Scn9a, NM_001290674.1), mouse 5HT3 (Htr3a, NM_001099644.1), and mouse TRPM8 (Trpm8, NM_134252.3) coding regions. We used the following primary antibodies: mouse anti-NF200 (1:10,000, Sigma), rabbit anti-CGRP (1:10,000, Peninsula Labs), and rabbit anti-TH (1:5,000, AbCam). We used fluorophore-conjugated secondary antibodies raised in goat against mouse or rabbit, as appropriate (1:1,000, Alexa Fluor 488, Life Technologies). To identify IB4-binding cells, biotinylated IB4 (1:1,000, Vector Labs) and fluorophore-conjugated streptavidin (1:1,000, Alexa Fluor 488, Life Technologies) were used in place of primary and secondary antibodies. Fos staining was performed 90 minutes after hindpaw injection of Hm1a or PBS. Spinal cord sections were prepared from lumbar L4/L5 and stained with rabbit anti-Fos (1:5,000, CalBiochem). ATF3 antibody (Santa Cruz Biotechnology) was used at 1:2000.

Statistics and Experimental Design.

Sample sizes for cellular physiology, histology and animal behavior were chosen based on previous experience with these assays as the minimum number of independent observation required for statistically significant results. For histology, at least three sections from each of at least three animals were counted. For oocyte and mouse neuron experiments, multiple batches/litters were used for all experiments. For behavioral experiments, animals were randomly chosen for different experimental cohorts by a blinded experimenter. Experimental and control conditions were compared within the same experimental time-course using randomly selected animals from one or multiple cages. Responses were then scored by an experimenter blinded to injection condition and experimental cohort. Animal genotype was tracked by ear tags and genotype unblinding occurred after analysis was complete.

Data were analyzed using Prism 6 software (GraphPad Software, San Diego, Calif., USA) and significance testing used either Student's t-tests or one-way analysis of variance (ANOVA) followed by Bonferroni or Tukey's post-hoc tests, as noted in legends. All significance tests are two-sided. Significance levels are *p<0.05, **p<0.01, ***p<0.001, and ****p<0.0001. The number of experiments (n) and significance are reported in the figure legends. All significance tests were justified as appropriate given the experimental design and nature of the comparisons. We assume equal variance and normally distributed data within experimental paradigms where comparisons are made. These are common assumptions relied upon for significance testing within these experimental paradigms as previously published by our group and others.

Behavior.

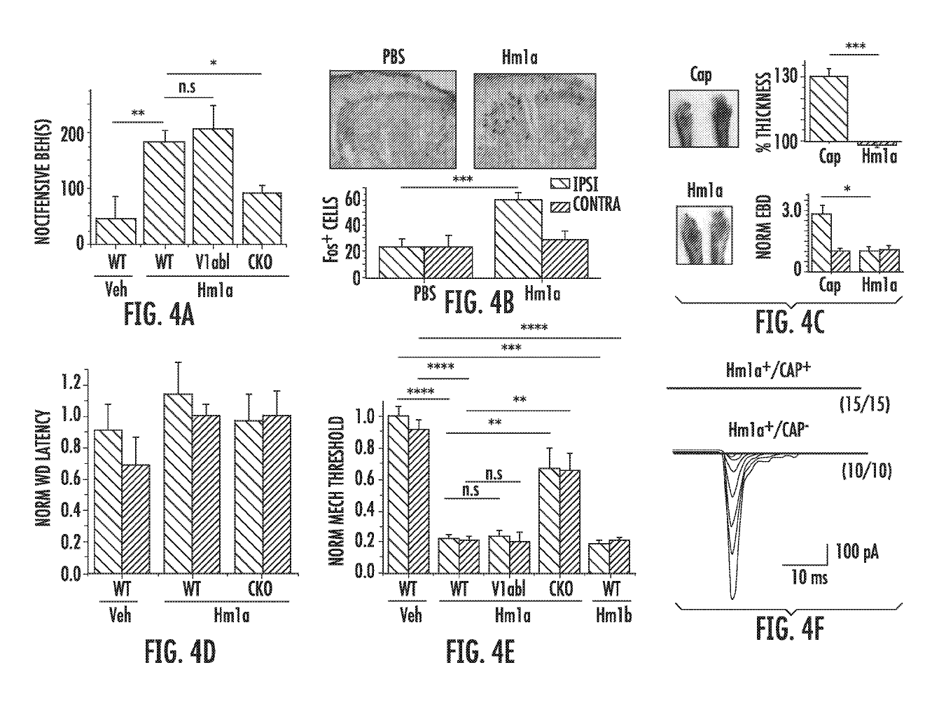

For behavioral experiments in FIG. 4, adult mice (6-12 weeks) were used. Male and female mice were first considered separately in hindpaw nocifensive response experiments. Both sexes showed significantly greater responses to toxin in WT littermate versus Naᵥ1.1^{F/F}×Per-Cre CKO mice (one-sided unpaired Student's t test, p<0.05, WT female: n=5, CKO female: n=6, WT male: n=5, CKO male: n=5). Therefore, male and female behavioral responses were pooled and subsequent experiments were performed on both male and female mice for CKO and WT littermate experiments, or only male mice for other conditions (e.g. Cap ablation). Nocifensive responses were recorded during a 20 minute observation period immediately following intraplantar injections (10 μl PBS with or without 5 μM Hm1a). Licking/biting behavior was scored as seconds of behavior with the experimenter blinded to injection condition and experimental cohort (WT, CKO or Cap Ablated mice). Hargreaves and Von Frey tests were performed 30 minutes after intraplantar injection of 500 nM Hm1a or Hm1b. I.t. cap ablation was performed as previously described³⁴, and i.t. cap treated mice were tested on a hot plate to ensure ablation of TRPV1+afferents. Ablation was also confirmed by histology.

Model of Chronic Visceral Hypersensitivity

Colitis was induced by administration of TNBS as described previously^{62,63}. Briefly, 13 week old anaesthetized mice were administered an intra-colonic enema of 0.1 mL TNBS (130 μg/mL in 30% EtOH) via a polyethylene catheter^{62,63,66}. Histological examination of mucosal architecture, cellular infiltrate, crypt abscesses, and goblet cell depletion confirmed significant TNBS-induced damage by day 3 post-treatment, which largely recovered by day 7, and fully recovered by 28 days. High-threshold nociceptors from mice at the 28-day time point displayed significant mechanical hypersensitivity, lower mechanical activation thresholds, and hyperalgesia and allodynia⁶⁷. As such, they are termed ‘chronic visceral hypersensitivity’ (CVH) mice^{62,63,66,68}.

Retrograde Tracing and Cell Culture of Colonic DRG Neurons.

Healthy and CVH mice of 16 weeks of age were anesthetized with halothane and following midline laparotomy, three 10 μL injections of the fluorescent retrograde neuronal tracer cholera toxin subunit B conjugated to AlexaFluor-488 were made sub-serosally within the wall of the descending colon. Four days after injection mice were sacrificed by CO₂ inhalation and DRGs from T10-L1 were surgically removed. DRGs were digested with 4 mg/mL collagenase II (GIBCO, Invitrogen) and 4 mg/mL dispase (GIBCO) for 30 min at 37° C., followed by 4 mg/mL collagenase II for 10 min at 37° C. Neurons were mechanically dissociated into a single-cell suspension via trituration through fire-polished Pasteur pipettes. Neurons were resuspended in DMEM (GIBCO) containing 10% FCS (Invitrogen), 2mM L-glutamine (GIBCO), 100 μM MEM non-essential amino acids (GIBCO) and 100 mg/ml penicillin/streptomycin (Invitrogen). Neurons were spot-plated on 8 mm HCl treated coverslips coated with poly-D-lysine (800 μg/ml) and laminin (20 μg/ml) and maintained in an incubator at 37° C. in 5% CO₂.

### Example 1

Venom Screen Identifies Selective Naᵥ1.1 Activating Toxins.

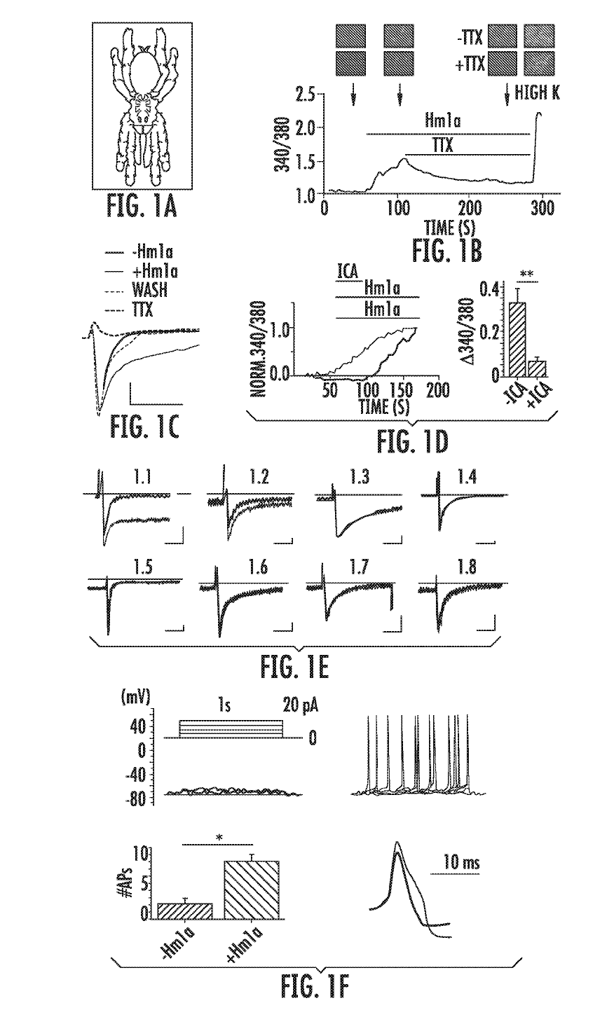

To identify novel toxins that target primary afferent nociceptors, we used calcium imaging to screen a collection of 109 spider, scorpion and centipede venoms for the ability to activate cultured somatosensory neurons. Venom from the tarantula Heteroscodra maculata (FIG. 1a) robustly excites a subset of neurons from trigeminal ganglia (TG) or dorsal root ganglia (DRG) from mice or rats. Venom fractionation yielded two active peaks, which were identified by MALDI-MS and Edman sequencing. We named these toxins δ-theraphotoxin-Hm1a (Hm1a) and δ-theraphotoxin-Hm1b (Hm1b), two inhibitor cysteine knot (ICK) peptides of related sequence. Applying synthetic Hm1a to rat DRG neurons likewise triggers calcium responses (FIG. 1b), validating Hm1a as an active venom component. All subsequent experiments were performed with synthetic Hm1a peptide unless otherwise stated.

Tetrodotoxin (TTX) blocked Hm1a-evoked calcium responses (FIG. 1b), suggesting involvement of Naᵥ channels. Indeed, whole-cell patch-clamp recordings from TG neurons show that Hm1a robustly inhibits Naᵥ current inactivation (FIG. 1c). Somatosensory neurons express several Naᵥ channel subtypes, including Naᵥ1.1, 1.6, 1.7, 1.8, and 1.9; however only Naᵥ1.1, 1.6 and 1.7 are sensitive to TTX²¹, thus narrowing our search. We next tested ICA-121431, a small molecule inhibitor with selectivity for Naᵥ1.1 and Naᵥ1.3²² (FIG. 1b), and found that it greatly diminishes Hm1a-evoked calcium responses in both embryonic DRG and P0 mouse TG cultures (FIG. 1d), suggesting that Naᵥ1.1 is the main target among the major sensory neuron subtypes, In contrast, ICA-121431 only partially blocks responses to SGTx1, an Hm1a-related peptide that shows little selectivity among Naᵥ channel subtypes. As such, it is not surprising that SGTx1 excites a larger cohort of TG and embryonic DRG neurons than Hm1a (FIGS. 1c-d). To confirm toxin selectivity for Naᵥ1.1, we heterologously expressed Naᵥ1.1-1.8 channels in Xenopus oocytes. Remarkably, Hm1a inhibits hNaᵥ1.1 fast inactivation (EC₅₀=38±6 nM), with substantially weaker effects on hNaᵥ1.2 and hNaᵥ1.3, and no effect on hNaᵥ1.4 -1.8 (FIG. 1e). [Similar results were obtained with native Hm1b.] Naᵥ1.9 is not efficiently expressed in recombinant systems, but surrogate chimeras (rKᵥ2.1 channels containing the S3b-S4 toxin-binding region from each of the four hNaᵥ1.9 domains) were also toxin insensitive.

Hm1b is a novel toxin, but Hm1a was previously described as ic-theraphotoxin-Hm1a moderate-affinity blocker of Kᵥ4.1 voltage-gated potassium (Kᵥ) channels²⁵. We find, however, that high concentrations (up to 5 μM) of synthetic Hm1 a blocks <20% of mKᵥ4.1 current. We found that 1 μM native Hm1a displays a saturating effect on Naᵥ1.1, but likewise fails to block mKᵥ4.1. Finally, in cultured sensory neurons, outward K⁺ currents were unaffected by 500 nM Hm1a, thus suggesting that its main physiologic target is Naᵥ1.1. The effect on Naᵥ1.1 may also explain why injection of Hm1a into the brain was previously shown to elicit convulsions and rapid death. Taken together, these results demonstrate that Hm1 a activates a subset of sensory neurons by selectively targeting Naᵥ1.1.

Inhibition of Naᵥ channel fast inactivation should render cells hyperexcitable without directly altering resting membrane potential. Indeed, analysis of Hm1 a-responsive TG neurons in whole-cell current clamp configuration shows this to be the case. Hm1 a does not alter resting membrane potential (before Hm1a, Vm=−55±6 mV; after Hm1a, Vm=−56±6 mV); however, it robustly enhances spike frequency during a 20 pA current injection. Hm1a also significantly prolongs the action potential width by ˜28%, consistent with the introduction of non-inactivating Na current (FIG. 10. In the absence of direct effects on membrane voltage, toxin-evoked calcium signals would depend on “spontaneous” cellular depolarization. In fact, we found that toxin responses were most robust in sensory neuron cultures derived from young (embryonic or newborn) mice or rats, likely reflecting a lower threshold for action potential firing in these cells or culture conditions. Consistent with this hypothesis, we found that prostaglandin E2 (PGE₂) sensitization²⁶ of adult neurons prior to toxin exposure greatly enhances the percentage of toxin sensitive cells.

### Example 2

Hm1a Selectivity Depends on the S1-S2 loop in DIV of Naᵥ1.1.

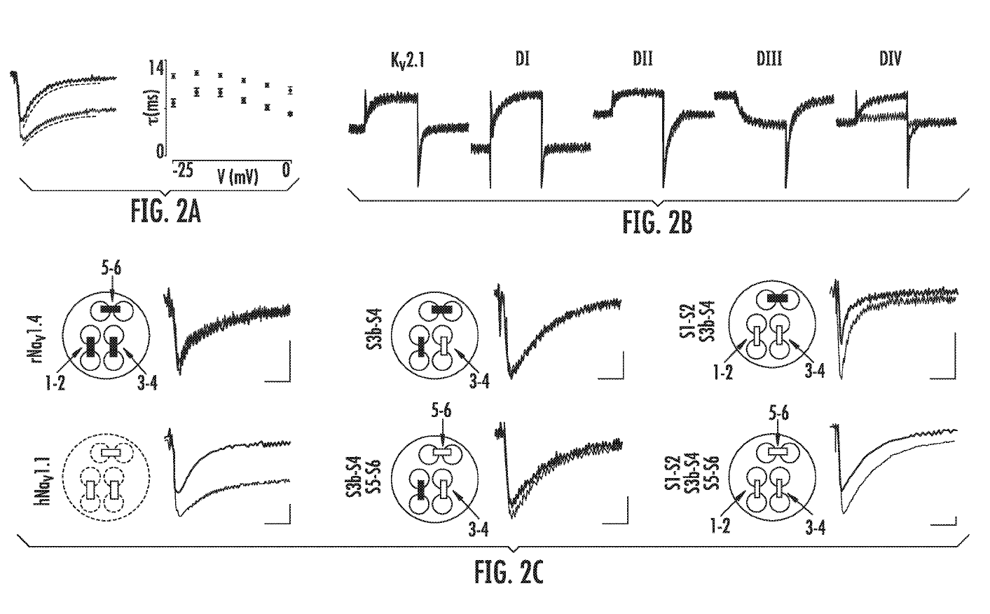

Analysis of Hm1a effects reveals that the toxin inhibits both the speed and extent of fast inactivation (FIG. 2a), similar to the mechanism described for less selective peptide toxins that bind to the S3b-S4 voltage sensor region of Domain IV (DIV)²³. To determine whether Hm1a targets the same locale, we transferred each of four S3b-S4 regions from hNaᵥ1.1 into the cognate location of the homo-tetrameric rKᵥ2.1 channel, which is normally insensitive to the toxin. Indeed, transfer of just the DIV S3b-S4 region renders rKᵥ2.1 susceptible to Hm1a, demonstrating that this segment is a primary determinant of toxin action (FIG. 2b). However, this region is identical or highly conserved in hNaᵥ1.1, 1.2 and 1.3, and thus while the toxin may interact with DIV S3b-S4, such interaction does not fully account for toxin selectivity. To identify additional regions that specify toxin selectivity, we constructed chimeras between Naᵥ1.1 and Naᵥ1.4, the latter being completely insensitive to Hm1a. Replacement of the S3b-S4 region from hNaᵥ1.4 with that of hNaᵥ1.1 did not confer toxin sensitivity, whereas transfer of both the S3b-S4 region plus the S1-S2 loop from Naᵥ1.1 resulted in toxin sensitivity (FIG. 2c). These results suggest that both the S1-S2 loop and S3b-S4 region together determine toxin susceptibility and subtype selectivity, consistent with previous suggestions that the S1-S2 loop can contribute to toxin recognition sites on voltage sensors.

### Example 3

Naᵥ1.1 is not Expressed in Classic C-Fiber Nociceptors.

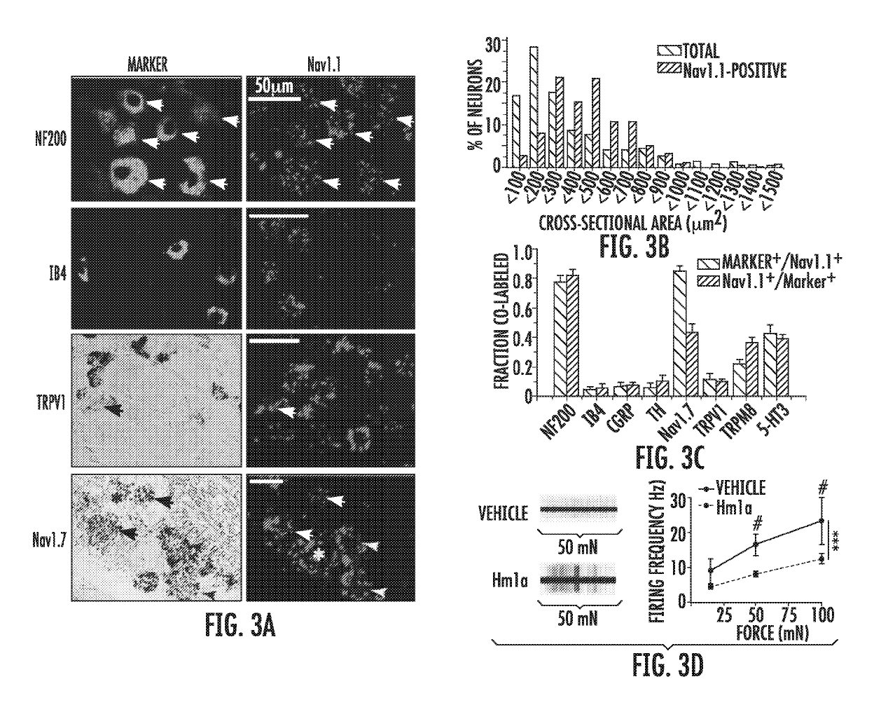

Previous studies have shown that Naᵥ1.1 is expressed by medium and large diameter, myelinated sensory neurons⁷, consistent with our data showing selective enrichment of Naᵥ1.1 transcripts in medium diameter (cross-sectional area 400-700 μm²) neurons in adult mouse DRG (FIG. 3). We detected Naᵥ1.1 mRNA in 35% of all cells, most of which (>75%) belong to the myelinated (NF200-positive) cohort. In contrast, we observed limited (5-11%) overlap of Naᵥ1.1-positive cells with markers of small diameter, unmyelinated neurons, including TRPV1, CGRP, tyrosine hydroxylase and the lectin IB4. However, we did see substantial co-expression with the 5-HT₃ receptor (43% of Naᵥ1.1-positive cells express 5-HT₃), a serotonin-gated channel that is expressed primarily by lightly myelinated Aδ neurons²⁹. Finally, 22% of Naᵥ1.1-positive cells also expressed the cold/menthol receptor, TRPM8, which is found in both C and Aδ fibers³⁰. Taken together, we conclude that Naᵥ1.1 is expressed primarily by myelinated neurons, including Aδ fibers, consistent with published single-cell transcriptome profiling data from dissociated DRG neurons³¹. Interestingly, most (>85%) Naᵥ1.1-positive cells also express Naᵥ1.7, suggesting that this population of myelinated neurons may contribute to nociception (see below).

To complement this histological analysis, we also examined overlap of toxin sensitivity with that of other receptor-selective agonists. Hm1 a responders constitute 13% of TG neurons cultured from newborn (P0) mice, of which few (<13%) respond to mustard oil (AITC), an agonist of the C fiber-restricted TRPA1 receptor. Moreover, only a third of toxin-sensitive P0 neurons responds to capsaicin, despite the fact that a majority (˜60%) of sensory neurons expresses TRPV1 at this early stage³². Over half (52%) of toxin-sensitive cells responds to mCPBG, a selective 5-HT₃ agonist, while 38% of toxin responsive cells also reacts to menthol. Moreover, few if any of the toxin-sensitive cells binds IB4. Finally, we explored the effect of Hm1a on mechanonociceptive Aδ fibers (AM's) using the ex vivo skin-nerve preparation. We found that application of 1 μM Hm1a to cutaneous receptive fields significantly increases firing rate in these AM fibers during mechanical stimuli (FIG. 3d), thus confirming expression of functional Naᵥ1.1 in this fiber class. Previous studies have shown limited expression of TRPV1 in AM fibers³³, consistent with the above histological data showing only partial overlap between Naᵥ1.1 and TRPV1. Taken together, these functional data confirm our histological assignment of Naᵥ1.1 expression to myelinated Aδ fibers, and further suggest that this particular Naᵥ channel subtype participates in mechanical nociception.

### Example 4

Hm1a Elicits Non-Inflammatory Pain and Mechanical Allodynia.

We next used Hm1 a to directly ask whether activation of Naᵥ1.1-expressing fibers produces pain behaviors. Indeed, injection of Hm1a (5 μM in 10 μl) into the mouse hind paw elicits immediate and robust nocifensive responses (bouts of licking or biting of the injected paw) throughout the observation period (FIG. 4a). Toxin injection also significantly increases Fos immunoreactivity in dorsal horn neurons of the superficial lamina ipsilateral to the injection, signifying functional engagement of myelinated nociceptors and their central connections (FIG. 4b).

To exclude the possibility that toxin-evoked nociception depends on the small population of fibers co-expressing TRPV1 and Naᵥ1.1, we ablated TRPV1-positive terminals by intrathecal (spinal) injection of capsaicin, in which case Hm1a-evoked nocifensive behavior persisted (FIG. 4a). Remarkably, Hm1a does not produce swelling or plasma extravasation of the injected paw, a neurogenic inflammatory response readily provoked by activation of peptidergic C-fiber nociceptors that include most TRPV1-expressing neurons (FIG. 4c). These results, together with our histological and functional characterization of Naᵥ1.1 expression, further suggest that Hm1a elicits pain by activating a non-peptidergic subset of myelinated sensory fibers.

Genetic or pharmacologic elimination of TRPV1-expressing fibers greatly diminishes sensitivity to noxious heat, but does not perturb sensitivity to mechanical stimuli. In contrast, the anatomical and physiological results described above suggest that Naᵥ1.1-positive fibers contribute predominantly to mechanonociception. We therefore asked whether Hm1 a has differential effects on these behavioral modalities by monitoring responses to thermal and mechanical stimuli following intraplantar injection of toxin at a dose (500 nM in 10 μl) insufficient to elicit acute behavior. Indeed, intraplantar injection of Hm1a does not alter sensitivity to heat, but produces robust sensitization to mechanical stimulation that is not dependent on TRPV1-expressing fibers (FIG. 4d, e). Equivalent mechanical sensitization is also observed following injection of native Hm1b peptide (FIG. 4e). In agreement with these behavioral observations, we find that all Hm1 a-responsive adult DRG neurons display mechanically activated currents, except for those neurons that are also capsaicin sensitive (FIG. 4f).

To confirm the requirement of Naᵥ1.1 in toxin-evoked behaviors, we crossed mice bearing a floxed Naᵥ1.1 allele¹³ to a line that expresses Cre recombinase under control of the peripherin promoter, which is active in a large percentage of unmyelinated and myelinated sensory neurons during development. Indeed, analysis of a peripherin-Cre x YFP reporter line showed that these animals express Cre recombinase in 46% of Naᵥ1.1-positive cells. Strikingly, elimination of Naᵥ1.1 from this subset of fibers significantly attenuates toxin-evoked behaviors, including both acute nocifensive responses and mechanical sensitization (FIG. 4a, e).

Robust activation of nociceptive pathways by nerve injury or inflammation can trigger both primary and secondary sensitization, the latter of which can manifest as mechanical or heat hypersensitivity contralateral to the site of inflammation or injury. In fact, we find that unilateral injection of Hm1a produced robust and equivalent mechanical sensitization of both the injected and contralateral paw (FIG. 4e). This contralateral sensitization is also modality specific as no change in heat sensitivity was observed (FIG. 4d). Importantly, Hm1a-mediated mechanical sensitivity is equivalently reduced in ipsilateral and contralateral paws of Naᵥ1.1-peripherin Cre animals, demonstrating that contralateral effects depend on Naᵥ1.1 (FIG. 4e). Since we do not observe signs of neurogenic inflammation, we asked whether this phenotype results from Hm1 a-mediated nerve injury. However, this seems unlikely since toxin injection fails to induce expression of ATF3, a marker of nerve damage'. Taken together, these observations demonstrate that direct activation of Naᵥ1.1-expressing fibers is sufficient to produce robust and modality-specific bilateral sensitization.

### Example 5

Naᵥ1.1 is Upregulated in a Model of Irritable Bowel Syndrome.

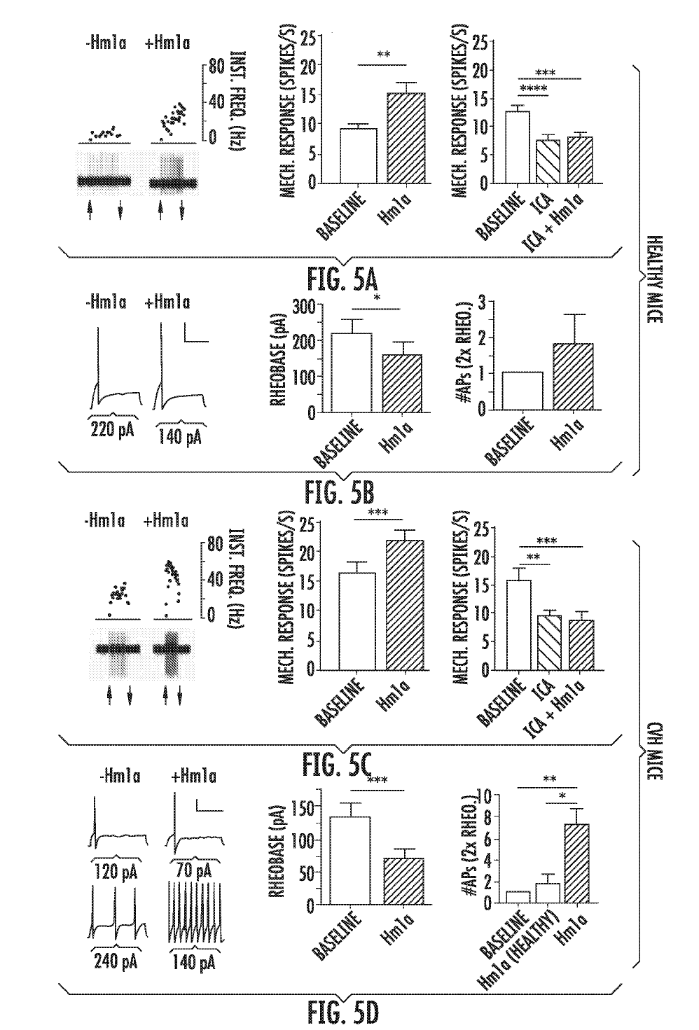

Chronic mechanical hypersensitivity underlies the development of abdominal pain in patients with irritable bowel syndrome (IBS). Given the apparent role of Naᵥ1.1 in mechano-nociception, we asked if this channel is expressed by mechanically sensitive fibers of the gut, and if so, whether it contributes to neuronal sensitization in a model of chronic visceral mechanical hypersensitivity (CVH)⁴². To address these questions, we examined mechanical responses in an ex vivo gut-nerve preparation from healthy or CVH mice. In preparations from healthy animals, Hm1a increases mechanically-evoked spiking in a sub-population (40%) of high-threshold colonic afferents that constitute presumptive mechano-nociceptors (FIG. 5a). Correspondingly, ICA-121431 reduces mechanical responses and blocks Hm1a sensitization in 50% of fibers examined (FIG. 5a). Moreover, Hm1a significantly reduces the threshold for action potential firing in a subset (45%) of retrogradely traced colonic DRG neurons as measured by whole-cell current clamp analysis (FIG. 5b). These results demonstrate that a subset of high-threshold mechanosensitive colonic fibers express functional Naᵥ1.1 channels.

In colonic afferents from CVH mice, baseline mechanosensory responses are elevated compared to healthy controls (compare FIGS. 5a and 5c). Application of Hm1a enhances mechanically-evoked spiking in a subset (36%) of CVH fibers beyond this already elevated level (FIG. 5c). Interestingly, in the context of CVH (and in contrast to normal controls), toxin application dramatically increases the electrical excitability of most (64%) retrogradely traced colonic DRG neurons (FIG. 5d), suggesting functional upregulation of Naᵥ1.1. Furthermore, ICA-121431 reduces mechanosensory responses in most (70%) CVH sensitized fibers to levels resembling those of baseline controls (compare FIGS. 5a and 5c), and blocks the sensitizing effects of Hm1a (FIG. 5c). Taken together, these results support a role for Naᵥ1.1 in mechanical hypersensitivity in IBS.

Development of Naᵥ channel subtype-selective ligands is an important, but challenging goal. Our results identifed sites within the DIV S1-S2 loop that enhance subtype selectivity, providing a useful strategy for designing other subtype-specific gating modifiers as shown below.

### Example 6

FB NaV1.1 blocker' reduces mechanosensitivity in a sub-population of colonic nociceptors from mice with CVH.

Ex vivo single-unit extracellular recordings of action potential discharge were made from splanchnic colonic afferents. Recordings were made from healthy or CVH mice using standard protocols (e.g. Brierley, S. M., Jones, R. C., III, Gebhart, G. F. & Blackshaw, L. A. Splanchnic and pelvic mechanosensory afferents signal different qualities of colonic stimuli in mice. Gastroenterology 127, 166-178 (2004)). Baseline mechanosensitivity was determined in response to application of a 2-g Von Frey hair probe to the afferent receptive field for 3 s. This process was repeated 3-4 times, separated each time by 10 s. Mechanosensitivity was then re-tested after the application of Hm1a (100 nM) or the Naᵥ1.1 blocker compound B (100 μM) or a combination thereof. Instantaneous frequency is defined as the inverse of the time interval between an action potential and the previous action potential. Group data are presented as spikes per second and are expressed as mean±s.e.m.

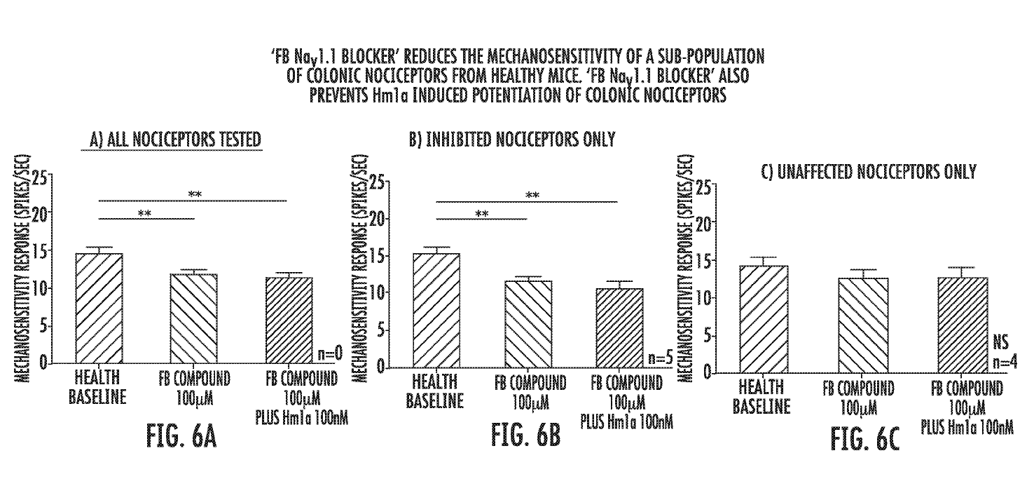

FIGS. 6A-6C show that Compound B at 100 μM concentration inhibits the mechanosensitivity of colonic nociceptors from healthy mice. Moreover, Compound B inhibits the sensitizing effect of Hm1a toxin on the neurons and still retains its inhibitory activity.

### Example 7

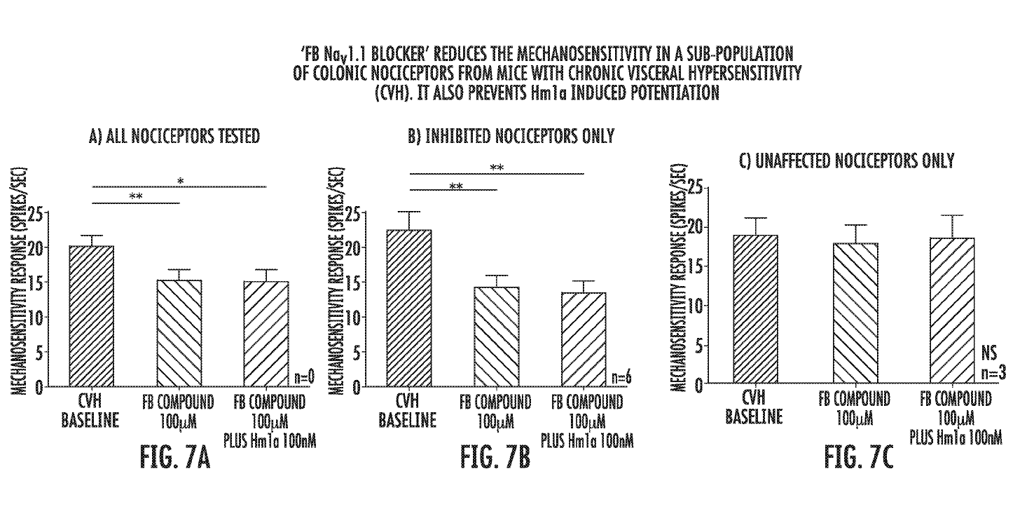

Using the same CVH model as described in Example 5, in colonic afferents from CVH mice, we tested whether Compound B would act as a Naᵥ1.1 channel blocker and inhibit baseline mechanosensory responses compared to healthy controls. As shown in FIG. 7A-7C, Compound B reduced mechanosensitivity in a sub-population of colonic nociceptors from CVH mice. As in Example 6, Compound B also was not affected by the presence of Hm1a and Hm1a blocked the stimulatory activity of Hm1a toxin.

The data show that the class of compounds, exemplified by Compound B are blockers of Naᵥ1.1 channels and can be used as a therapeutic composition for treatment of mechanosensitive neuron mediated pain and disease, such as pain associated with IBS.

### Example 8

Antiallodynic Effect of Compound B in the Nitroglycerin (NTG) Model for Migraine.

NTG-induced hind paw mechanical allodynia. Mechanical thresholds were determined with von Frey monofilaments (VFF; eight filaments, range 0.008-2 g, Stoelting Co) using the Dixon up-and-down method⁵¹. For drug testing, 48 naive C57Bl/6 male mice (20-30 g) were divided randomly to the following groups (n=12 mice per group): Vehicle/Cyclodextrin, Vehicle/Compound B 75 mg/kg, NTG/Cyclodextrin, and NTG/Compound B 75 mg/kg. Prior to testing all animals were handled for 1 week in the behavior room during mid-morning using the cupped hand technique⁵². On each testing day, a maximum of 12 animals was used, divided equally into the four groups. Mice were confined in clear acrylic cages (8.7″×8.7″×5″) divided into four chambers, each on a raised wire mesh platform that allowed full access to the tested paws. Mice were acclimated for two hours, on the day of testing and one day prior. Mechanical thresholds were evaluated before (baseline), and 75 and 120 min after i.p. administration of 10 mg/kg NTG (or vehicle), in accordance with NTG's time-to-peak-effect (TPE) in this model (data not shown). 40% Cyclodextrin in saline or 75 mg/kg Compound B dissolved in 40% cyclodextrin in saline, was administered contralateral to NTG administration thirty minutes before the first post-NTG time point, in accordance with compound B's TPE. Each filament was applied perpendicular to the center of each of the hind paw five times, spaced 1 sec apart, starting with the middle VFF (0.4 g). In the absence of a response, the next VFF in the series was applied until a response was witnessed. Response to VFF was recorded as an immediate withdrawal of the tested hind paw to the applied stimulus, with or without an observed licking behavior. The withdrawal threshold was quantified as the mean of both hind paws. The experimenter was blind to the treatment group.

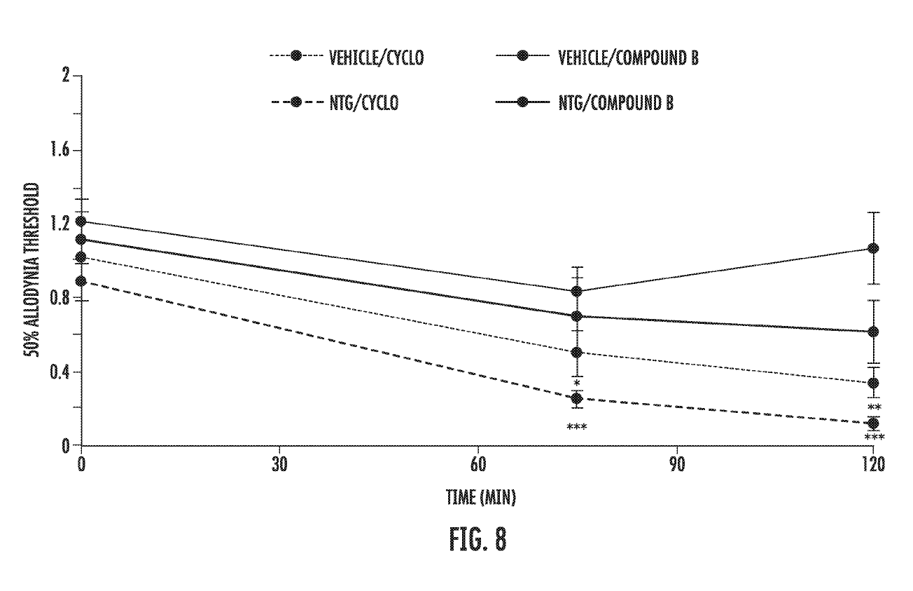

As shown in FIG. 8, there was no significant difference in allodynia threshold in the NTG/compound B (purple line) and Vehicle/Compound B (red line) groups compared to baseline threshold. Both the NTG/Cyclo (green line) and Vehicle/Cyclo (blue line) groups showed a reduction in mechanical allodynia at the 75 and 120 min time points after NTG/vehicle injection. These results indicate that Compound B was able to reverse NTG induced tactile allodynia and the mixture of Vehicle and cyclodextrin produced an allodynic effect. Thus, the data show that the class of compounds, exemplified by Compound B can be used as a therapeutic composition for treatment of mechanosensitive neuron mediated pain and disease, such as pain associated with migraine.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

1 Basbaum, A. I., Bautista, D. M., Scherrer, G. & Julius, D. Cellular and molecular mechanisms of pain. Cell 139, 267-284, doi:10.1016/j.cell.2009.09.028 (2009).
2 Dib-Hajj, S. D., Yang, Y., Black, J. A. & Waxman, S. G. The Na(V)1.7 sodium channel: from molecule to man. Nat Rev Neurosci 14, 49-62, doi:10.1038/nrn3404 (2013).
3 Faber, C. G. et al. Gain-of-function Naᵥ1.8 mutations in painful neuropathy. Proc Natl Acad Sci USA 109, 19444-19449, doi:10.1073/pnas.1216080109 (2012).
4 Fertleman, C. R. et al. SCN9A mutations in paroxysmal extreme pain disorder: allelic variants underlie distinct channel defects and phenotypes. Neuron 52, 767-774, doi:10.1016/j.neuron.2006.10.006 (2006).
5 Hoeijmakers, J. G., Faber, C. G., Merkies, I. S. & Waxman, S. G. Painful peripheral neuropathy and sodium channel mutations. Neurosci Lett 596, 51-59, doi:10.1016/j.neulet.2014.12.056 (2015).
6 Zhang, X. Y. et al. Gain-of-function mutations in SCN11A cause familial episodic pain. Am J Hum Genet 93, 957-966, doi:10.1016/j.ajhg.2013.09.016 (2013).
7 Fukuoka, T. et al. Comparative study of the distribution of the alpha-subunits of voltage-gated sodium channels in normal and axotomized rat dorsal root ganglion neurons. J Comp Neurol 510, 188-206, doi:10.1002/cne.21786 (2008).
8 Black, J. A., Liu, S. J., Tanaka, M., Cummins, T. R. & Waxman, S. G. Changes in the expression of tetrodotoxin-sensitive sodium channels within dorsal root ganglia neurons in inflammatory pain. Pain 108, 237-247, doi:10.1016/j.pian.2003.12.035 (2004).
9 Fukuoka, T., Miyoshi, K. & Noguchi, K. De Novo Expression of Nav1.7 in Injured Putative Proprioceptive Afferents: Multiple Tetrodotoxin-Sensitive Sodium Channels Are Retained in the Rat Dorsal Root after Spinal Nerve Ligation. Neuroscience 284, 693-706, doi:10.1016/j.neuroscience.2014.10.027 (2015).
10 Dib-Hajj, S. D., Cummins, T. R., Black, J. A. & Waxman, S. G. Sodium channels in normal and pathological pain. Annu Rev Neurosci 33, 325-347, doi:10.1146/annurev-neuro-060909-153234 (2010).
11 Wang, W., Gu, J., Li, Y. Q. & Tao, Y. X. Are voltage-gated sodium channels on the dorsal root ganglion involved in the development of neuropathic pain? Mol Pain 7, 16, doi:10.1186/1744-8069-7-16 (2011).
12 Catterall, W. A., Kalume, F. & Oakley, J. C. NaV1.1 channels and epilepsy. J Physiol 588, 1849-1859, doi:10.1113/jphysio1.2010.187484 (2010).
13 Cheah, C. S. et al. Specific deletion of NaV1.1 sodium channels in inhibitory interneurons causes seizures and premature death in a mouse model of Dravet syndrome. Proc Natl Acad Sci USA 109, 14646-14651, doi:10.1073/pnas.1211591109 (2012).
14 Han, S. et al. Autistic-like behaviour in Scnla+/− mice and rescue by enhanced GABA-mediated neurotransmission. Nature 489, 385-390, doi:10.1038/nature11356 (2012).
15 Verret, L. et al. Inhibitory interneuron deficit links altered network activity and cognitive dysfunction in Alzheimer model. Cell 149, 708-721, doi:10.1016/j.cell.2012.02.046 (2012).
16 Gargus, J. J. & Tournay, A. Novel mutation confirms seizure locus SCN1A is also familial hemiplegic migraine locus FHM3. Pediatr Neurol 37, 407-410, doi:10.1016/j.pediatrneuro1.2007.06.016 (2007).
17 Cestele, S., Schiavon, E., Rusconi, R., Franceschetti, S. & Mantegazza, M. Nonfunctional NaV1.1 familial hemiplegic migraine mutant transformed into gain of function by partial rescue of folding defects. Proc Natl Acad Sci USA 110, 17546-17551, doi:10.1073/pnas.1309827110 (2013).
18 Priest, B. T. Future potential and status of selective sodium channel blockers for the treatment of pain. Curr Opin Drug Discov Devel 12, 682-692 (2009).
19 Bohlen, C. J. & Julius, D. Receptor-targeting mechanisms of pain-causing toxins: How ow? Toxicon 60, 254-264, doi:10.1016/j.toxicon.2012.04.336 (2012).
20 Olivera, B. M., Hillyard, D. R., Marsh, M. & Yoshikami, D. Combinatorial peptide libraries in drug design: lessons from venomous cone snails. Trends Biotechnol 13, 422-426, doi:10.1016/S0167-7799(00)88996-9 (1995).
21 Black, J. A. et al. Spinal sensory neurons express multiple sodium channel alpha-subunit mRNAs. Brain Res Mol Brain Res 43, 117-131 (1996).
22 McCormack, K. et al. Voltage sensor interaction site for selective small molecule inhibitors of voltage-gated sodium channels. Proc Natl Acad Sci USA 110, E2724-2732, doi:10.1073/pnas.1220844110 (2013).
23 Bosmans, F., Martin-Eauclaire, M. F. & Swartz, K. J. Deconstructing voltage sensor function and pharmacology in sodium channels. Nature 456, 202-208, doi:10.1038/nature07473 (2008).
24 Bosmans, F., Puopolo, M., Martin-Eauclaire, M. F., Bean, B. P. & Swartz, K. J. Functional properties and toxin pharmacology of a dorsal root ganglion sodium channel viewed through its voltage sensors. J Gen Physiol 138, 59-72, doi:10.1085/jgp.201110614 (2011).
25 Escoubas, P., Diochot, S., Celerier, M. L., Nakajima, T. & Lazdunski, M. Novel tarantula toxins for subtypes of voltage-dependent potassium channels in the Kv2 and Kv4 subfamilies. Mol Pharmacol 62, 48-57, doi:UNSP 1508/992248. DOI 10.1124/mol.62.1.48 (2002).
26 Smith, J. A. M., Davis, C. L. & Burgess, G. M. Prostaglandin E-2-induced sensitization of bradykinin-evoked responses in rat dorsal root ganglion neurons is mediated by cAMP-dependent protein kinase A. Eur J Neurosci 12, 3250-3258, doi:DOI 10.1046/j.1460-9568.2000.00218.x (2000).
27 Wang, J. et al. Mapping the receptor site for alpha-scorpion toxins on a Na+ channel voltage sensor. Proc Natl Acad Sci USA 108, 15426-15431, doi:10.1073/pnas.1112320108 (2011).
28 Bende, N. S. et al. A distinct sodium channel voltage-sensor locus determines insect selectivity of the spider toxin Dc1a. Nat Commun 5, 4350, doi:10.1038/ncomms5350 (2014).
29 Zeitz, K. P. et al. The 5-HT3 subtype of serotonin receptor contributes to nociceptive processing via a novel subset of myelinated and unmyelinated nociceptors. Journal of Neuroscience 22, 1010-1019 (2002).
30 Bautista, D. M. et al. The menthol receptor TRPM8 is the principal detector of environmental cold. Nature 448, 204-208, doi:10.1038/nature05910 (2007).
31 Usoskin, D. et al. Unbiased classification of sensory neuron types by large-scale single-cell RNA sequencing. Nat Neurosci 18, 145-153, doi:10.1038/nn.3881 (2015).
32 Cavanaugh, D. J. et al. Restriction of Transient Receptor Potential Vanilloid-1 to the Peptidergic Subset of Primary Afferent Neurons Follows Its Developmental Downregulation in Nonpeptidergic Neurons. Journal of Neuroscience 31, 10119-10127, doi:10.1523/Jneurosci.1299-11.2011 (2011).
33 Stucky, C. L., Medler, K. A. & Molliver, D. C. The P2Y agonist UTP activates cutaneous afferent fibers. Pain 109, 36-44, doi:10.1016/j.pain.2004.01.007 (2004).
34 Cavanaugh, D. J. et al. Distinct subsets of unmyelinated primary sensory fibers mediate behavioral responses to noxious thermal and mechanical stimuli. Proc Natl Acad Sci USA 106, 9075-9080, doi:10.1073/pnas.0901507106 (2009).
35 Mishra, S. K., Tisel, S. M., Orestes, P., Bhangoo, S. K. & Hoon, M. A. TRPV1-lineage neurons are required for thermal sensation. EMBO J 30, 582-593, doi:10.1038/emboj.2010.325 (2011).
36 Mishra, S. K. & Hoon, M. A. Ablation of TrpV1 neurons reveals their selective role in thermal pain sensation. Molecular and Cellular Neuroscience 43, 157-163, doi:10.1016/j.mcn.2009.10.006 (2010).
37 Scherrer, G. et al. VGLUT2 expression in primary afferent neurons is essential for normal acute pain and injury-induced heat hypersensitivity. P Natl Acad Sci USA 107, 22296-22301, doi:10.1073/pnas.1013413108 (2010).
38 Koltzenburg, M., Wall, P. D. & McMahon, S. B. Does the right side know what the left is doing? Trends Neurosci 22, 122-127, doi:Doi 10.1016/S0166-2236(98)01302-2 (1999).
39 Kissin, I., Lee, S. S. & Bradley, E. L. Effect of prolonged nerve block on inflammatory hyperalgesia in rats—Prevention of late hyperalgesia. Anesthesiology 88, 224-232, doi:Doi 10.1097/00000542-199801000-00031 (1998).
40 Tsujino, H. et al. Activating transcription factor 3 (ATF3) induction by axotomy in sensory and motoneurons: A novel neuronal marker of nerve injury. Mol Cell Neurosci 15, 170-182, doi:10.1006/mcne.1999.0814 (2000).
41 Brierley, S. M. & Linden, D. R. Neuroplasticity and dysfunction after gastrointestinal inflammation. Nat Rev Gastroenterol Hepatol 11, 611-627, doi:10.1038/nrgastro.2014.103 (2014).
42 de Araujo, A. D. et al. Selenoether oxytocin analogues have analgesic properties in a mouse model of chronic abdominal pain. Nature Communications 5, doi:ARTN 3165. 10.1038/ncomms4165 (2014).
43 Jensen, H. S., Grunnet, M. & Bastlund, J. F. Therapeutic potential of Na(V)1.1 activators. Trends Pharmacol Sci 35, 113-118, doi:10.1016/j.tips.2013.12.007 (2014).
44 Khan, G. M., Chen, S. R. & Pan, H. L. Role of primary afferent nerves in allodynia caused by diabetic neuropathy in rats. Neuroscience 114, 291-299 (2002).
45 Tsunozaki, M. et al. A ‘toothache tree’ alkylamide inhibits A delta mechanonociceptors to alleviate mechanical pain. J Physiol-London 591, 3325-3340, doi:10.1113/jphysio1.2013.252106x (2013).
46 Pappagallo, M. Newer antiepileptic drugs: Possible uses in the treatment of neuropathic pain and migraine. Clin Ther 25, 2506-2538, doi:Doi 10.1016/S0149-2918(03)80314-4 (2003).
47 Calabresi, P., Galletti, F., Rossi, C., Sarchielli, P. & Cupini, L. M. Antiepileptic drugs in migraine: from clinical aspects to cellular mechanisms. Trends Pharmacol Sci 28, 188-195, doi:10.1016/j.tips.2007.02.005 (2007).
48 Gilchrist, J. et al. Naᵥ1.1 modulation by a novel triazole compound attenuates epileptic seizures in rodents. ACS Chem Biol 9, 1204-1212, doi:10.1021/cb500108p (2014).
49 Suter, M. R., Kirschmann, G., Laedermann, C. J., Abriel, H. & Decosterd, I. Rufinamide attenuates mechanical allodynia in a model of neuropathic pain in the mouse and stabilizes voltage-gated sodium channel inactivated state. Anesthesiology 118, 160-172, doi:10.1097/ALN.0b013e318278cade (2013).
50 Herzig, V. et al. ArachnoServer 2.0, an updated online resource for spider toxin sequences and structures. Nucleic Acids Res 39, D653-657, doi:10.1093/nar/gkq1058 (2011).
51 Chaplan, S. R., et al., Quantitative assessment of tactile allodynia in the rat paw. J Neurosci Methods, 1994. 53(1): p. 55-63.
52 Hurst, J. L. and R. S. West, Taming anxiety in laboratory mice. Nat Methods, 2010. 7(10): p. 825-6.

1.-11. (canceled)

12. A method for inhibition of mechanical nociceptors on myelinated neurons of a subject, comprising administering to the subject, an effective amount of a composition comprising one or more Naᵥ1.1 channel blockers of formula I: 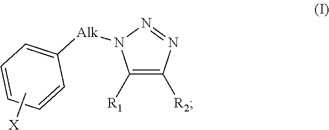
or a salt, solvate, or stereoisomer thereof, wherein X is H, or one or more electron withdrawing groups such as a halogen, NH₂, NO₂, SO₂, CN, or a C₁-C₆ alkyl group; Alk is C₁-C₃ alkyl; R₁ is H, C₁-C₆ alkyl, which may be substituted with OH, NH₂, alkylamino, amido, acyl, sulfonyl, sulfonylamino, and cyano groups; and R₂, is C₁-C₆ alkyl, alkenyl, and phenyl, which may be substituted with one or more OH, NH₂, alkylamino, amido, acyl, carboxyl, methoxyl, sulfonyl, and cyano groups.

13. The method of claim 12, wherein the one or more Naᵥ1.1 channel blockers of formula I are selected from the group consisting of: 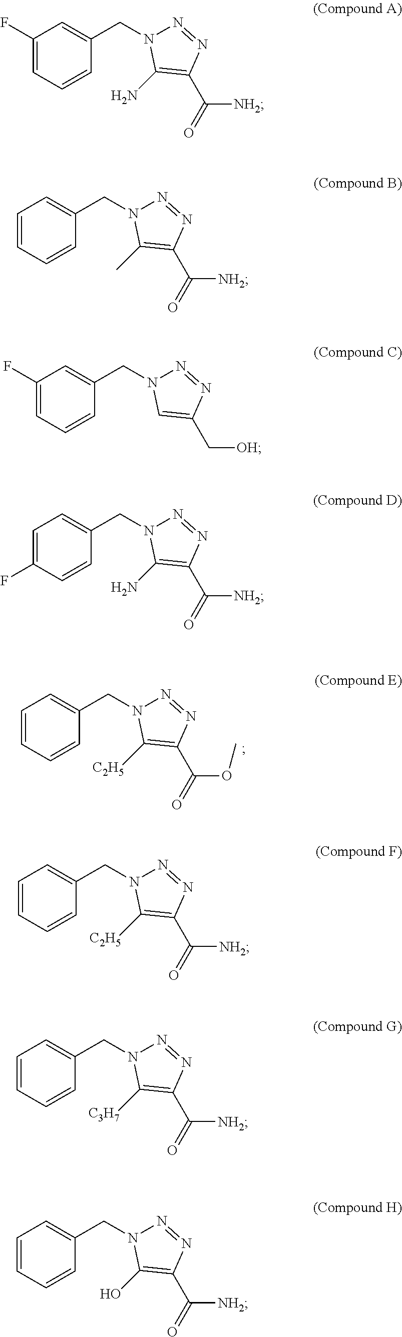 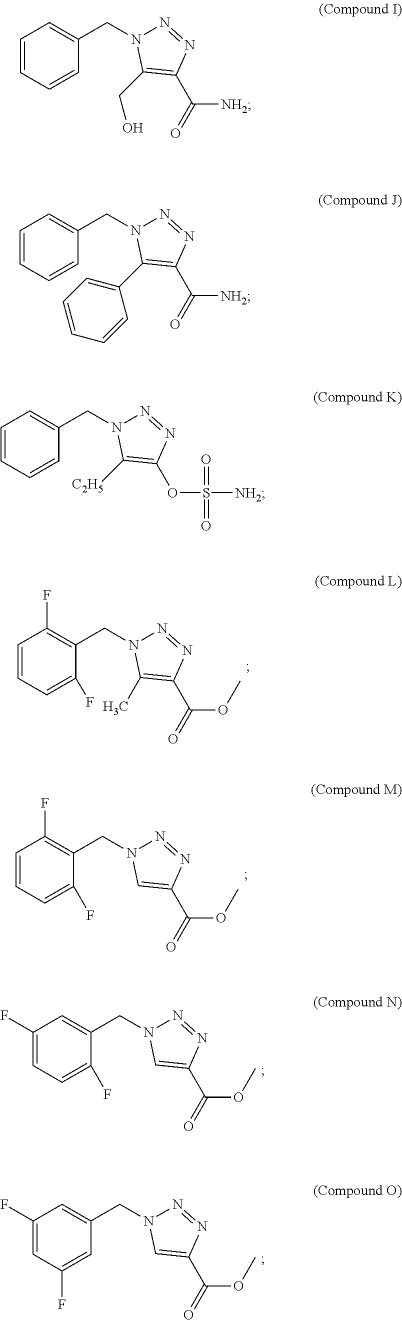 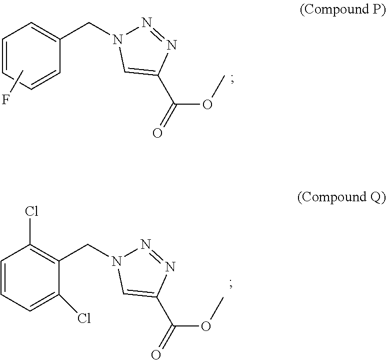
or a salt, solvate, or stereoisomer thereof.

14. The method of claim 13, wherein the one or more Naᵥ1.1 channel blockers of formula I are administered in conjunction with an effective amount of one or more additional biologically active agents.

15. The method of claim 14, wherein the one or more additional biologically active agents comprise enzymes, receptor antagonists or agonists, hormones and antibodies, autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, a-blocker sympatholytics, sympatholytics, sympathomimetics, adrenergic agonist sympathomimetics, intravenous anesthetics, barbiturate intravenous anesthetics, benzodiazepine intravenous anesthetics, opiate agonist intravenous anesthetics, skeletal muscle relaxants, neuromuscular blocker skeletal muscle relaxants, reverse neuromuscular blocker skeletal muscle relaxants, neurological agents, anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, antidepressants, heterocyclic antidepressants, monoamine oxidase inhibitors, selective serotonin re-uptake inhibitors, tricyclic antidepressants, antimanics, anti-psychotics, phenothiazine antipsychotics, anxiolytics, sedatives, hypnotics, barbiturate sedatives, benzodiazepine anxiolytics, sedatives, and hypnotics, and psychostimulants.

16. The method of claim 12, wherein the neurologic disease is selected from the group consisting of: febrile epilepsy, GEFS+, Dravet syndrome (also known as severe myclonic epilepsy of infancy or SMEI), borderline SMEI (SMEB), West syndrome (also known as infantile spasms), Doose syndrome (also known as myoclonic astatic epilepsy), intractable childhood epilepsy with generalized tonic-clonic seizures (ICEGTC), Panayiotopoulos syndrome, familial autism, Rasmussens's encephalitis and Lennox-Gastaut syndrome, Alzheimer's, migraine, including FHM3, the treatment of acute and/or chronic pain associated with mechanosensitive neuronal fibers in disorders including, Irritable Bowel Syndrome, static, mechanical or dynamic allodynias associated with neuropathies, complex regional pain syndrome, postherpetic neuralgia, fibromyalgia, spinal cord injury, menstrual cramps and related diseases.

17. A method for inhibition of mechanical pain in a subject suffering from a neurological disorder, comprising administering to the subject, an effective amount of a composition comprising one or more Naᵥ1.1 channel blockers of formula I: 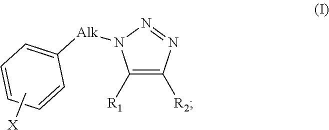
or a salt, solvate, or stereoisomer thereof, wherein X is H, or one or more electron withdrawing groups such as a halogen, NH₂, NO₂, SO₂, CN, or a C₁-C₆ alkyl group; Alk is C₁-C₃ alkyl; R₁ is H, C₁-C₆ alkyl, which may be substituted with OH, NH₂, alkylamino, amido, acyl, sulfonyl, sulfonylamino, and cyano groups; and R₂, is C₁-C₆ alkyl, alkenyl, and phenyl, which may be substituted with one or more OH, NH₂, alkylamino, amido, acyl, carboxyl, methoxyl, sulfonyl, and cyano groups.

18. The method of claim 17, wherein the one or more Naᵥ1.1 channel blockers of formula I are selected from the group consisting of: 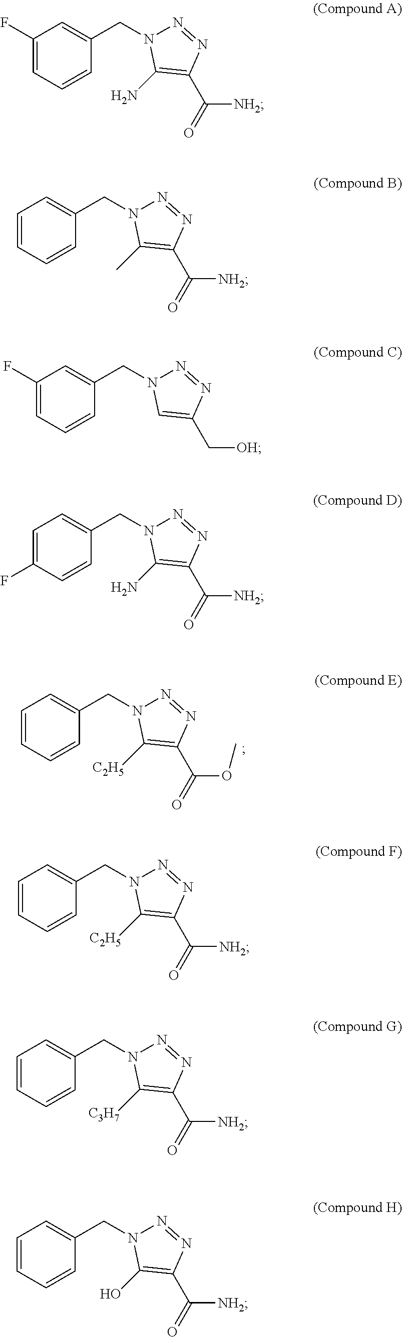 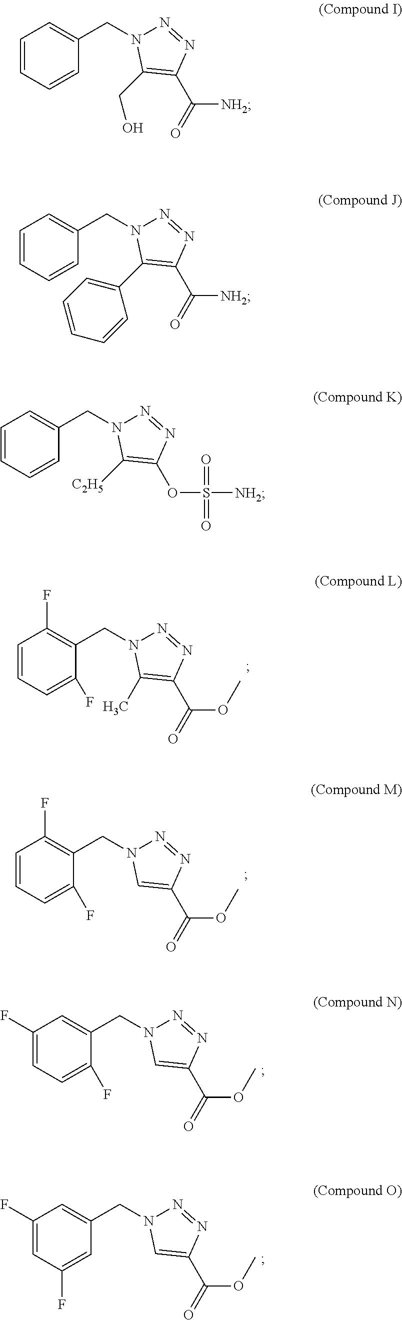 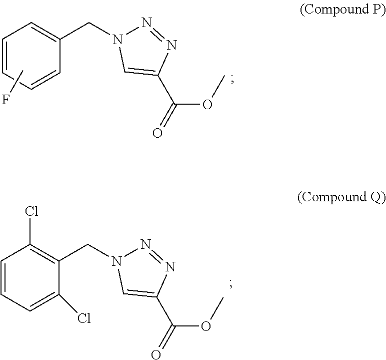
or a salt, solvate, or stereoisomer thereof.

19. The method of claim 18, wherein the one or more Naᵥ1.1 channel blockers of formula I are administered in conjunction with an effective amount of one or more additional biologically active agents.

20. The method of claim 19, wherein the one or more additional biologically active agents comprise enzymes, receptor antagonists or agonists, hormones and antibodies, autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, sympatholytics, sympathomimetics, adrenergic agonist sympathomimetics, intravenous anesthetics, barbiturate intravenous anesthetics, benzodiazepine intravenous anesthetics, opiate agonist intravenous anesthetics, skeletal muscle relaxants, neuromuscular blocker skeletal muscle relaxants, reverse neuromuscular blocker skeletal muscle relaxants, neurological agents, anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, antidepressants, heterocyclic antidepressants, monoamine oxidase inhibitors, selective serotonin re-uptake inhibitors, tricyclic antidepressants, antimanics, anti-psychotics, phenothiazine antipsychotics, anxiolytics, sedatives, hypnotics, barbiturate sedatives, benzodiazepine anxiolytics, sedatives, and hypnotics, and psychostimulants.

21. The method of claim 17, wherein the neurologic disease is selected from the group consisting of: febrile epilepsy, GEFS+, Dravet syndrome (also known as severe myclonic epilepsy of infancy or SMEI), borderline SMEI (SMEB), West syndrome (also known as infantile spasms), Doose syndrome (also known as myoclonic astatic epilepsy), intractable childhood epilepsy with generalized tonic-clonic seizures (ICEGTC), Panayiotopoulos syndrome, familial autism, Rasmussens's encephalitis and Lennox-Gastaut syndrome, Alzheimer's, migraine, including FHM3, the treatment of acute and/or chronic pain associated with mechanosensitive neuronal fibers in disorders including, Irritable Bowel Syndrome, static, mechanical or dynamic allodynias associated with neuropathies, complex regional pain syndrome, postherpetic neuralgia, fibromyalgia, spinal cord injury, menstrual cramps and related diseases.

22. A method for inhibition of splanchnic colonic afferent neurons of a subject suffering from Irritable Bowel Syndrome (IBS), comprising administering to the subject, an effective amount of a composition comprising one or more Naᵥ1.1 channel blockers of formula I: 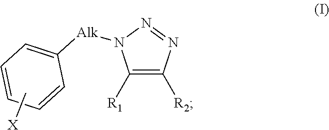
or a salt, solvate, or stereoisomer thereof, wherein X is H, or one or more electron withdrawing groups such as a halogen, NH₂, NO₂, SO₂, CN, or a C₁-C₆ alkyl group; Alk is C₁-C₃ alkyl; R₁ is H, C₁-C₆ alkyl, which may be substituted with OH, NH₂, alkylamino, amido, acyl, sulfonyl, sulfonylamino, and cyano groups; and R₂, is C₁-C₆ alkyl, alkenyl, and phenyl, which may be substituted with one or more OH, NH₂, alkylamino, amido, acyl, carboxyl, methoxyl, sulfonyl, and cyano groups.

23. The method of claim 22, wherein the one or more Naᵥ1.1 channel blockers of formula I are selected from the group consisting of: 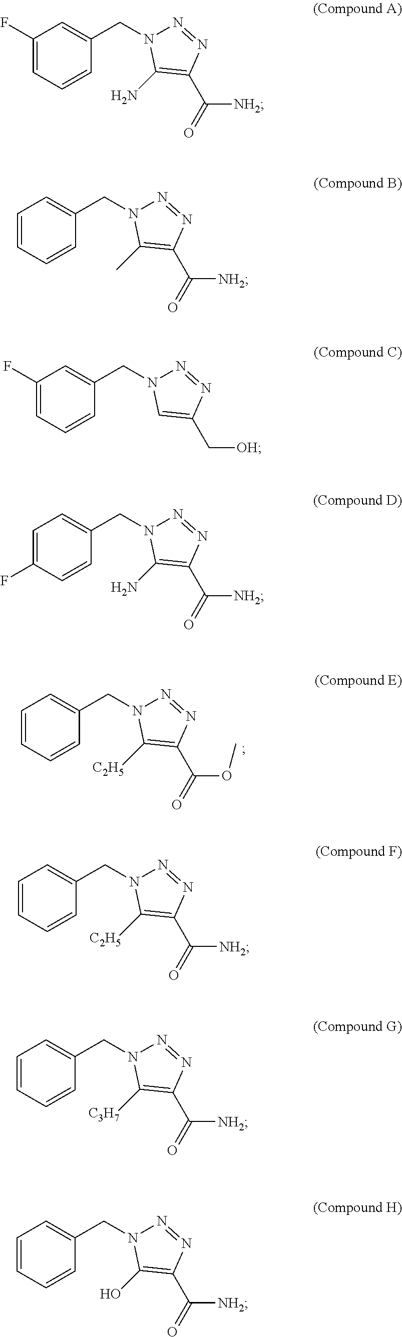 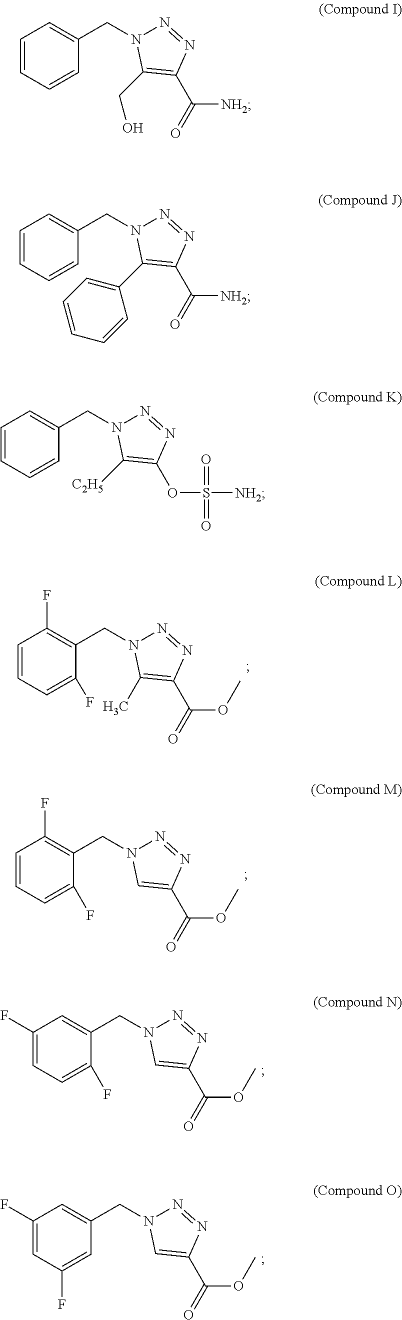 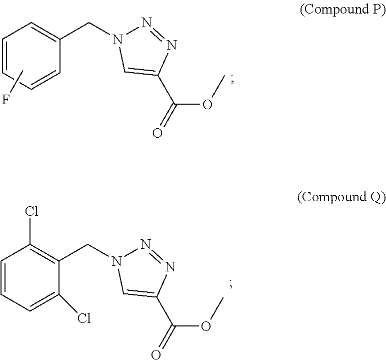
or a salt, solvate, or stereoisomer thereof.

24. The method of claim 22, wherein the one or more Naᵥ1.1 channel blockers of formula I are administered in conjunction with an effective amount of one or more additional biologically active agents.

25. The method of claim 24, wherein the one or more additional biologically active agents comprise enzymes, receptor antagonists or agonists, hormones and antibodies, autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, sympatholytics, sympathomimetics, adrenergic agonist sympathomimetics, intravenous anesthetics, barbiturate intravenous anesthetics, benzodiazepine intravenous anesthetics, opiate agonist intravenous anesthetics, skeletal muscle relaxants, neuromuscular blocker skeletal muscle relaxants, reverse neuromuscular blocker skeletal muscle relaxants, neurological agents, anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, antidepressants, heterocyclic antidepressants, monoamine oxidase inhibitors, selective serotonin re-uptake inhibitors, tricyclic antidepressants, antimanics, anti-psychotics, phenothiazine antipsychotics, anxiolytics, sedatives, hypnotics, barbiturate sedatives, benzodiazepine anxiolytics, sedatives, and hypnotics, and psychostimulants.

26. A method for treating Irritable Bowel Syndrome (IBS) in a subject suffering from IBS, or pain associated with IBS, comprising administering to the subject, an effective amount of a composition comprising one or more Nad .1 channel blockers of formula I: 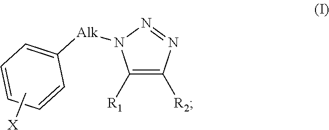
or a salt, solvate, or stereoisomer thereof, wherein X is H, or one or more electron withdrawing groups such as a halogen, NH₂, NO₂, SO₂, CN, or a C₁-C₆ alkyl group; Alk is C₁-C₃ alkyl; R₁ is H, C₁-C₆ alkyl, which may be substituted with OH, NH₂, alkylamino, amido, acyl, sulfonyl, sulfonylamino, and cyano groups; and R₂, is C₁-C₆ alkyl, alkenyl, and phenyl, which may be substituted with one or more OH, NH₂, alkylamino, amido, acyl, carboxyl, methoxyl, sulfonyl, and cyano groups.

27. The method of claim 26, wherein the one or more Naᵥ1.1 channel blockers of formula I are selected from the group consisting of: 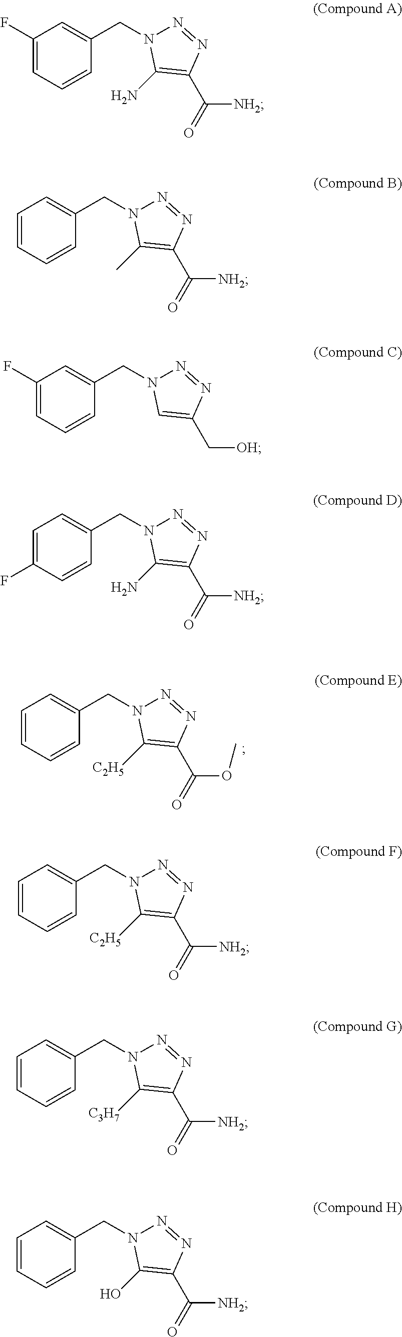 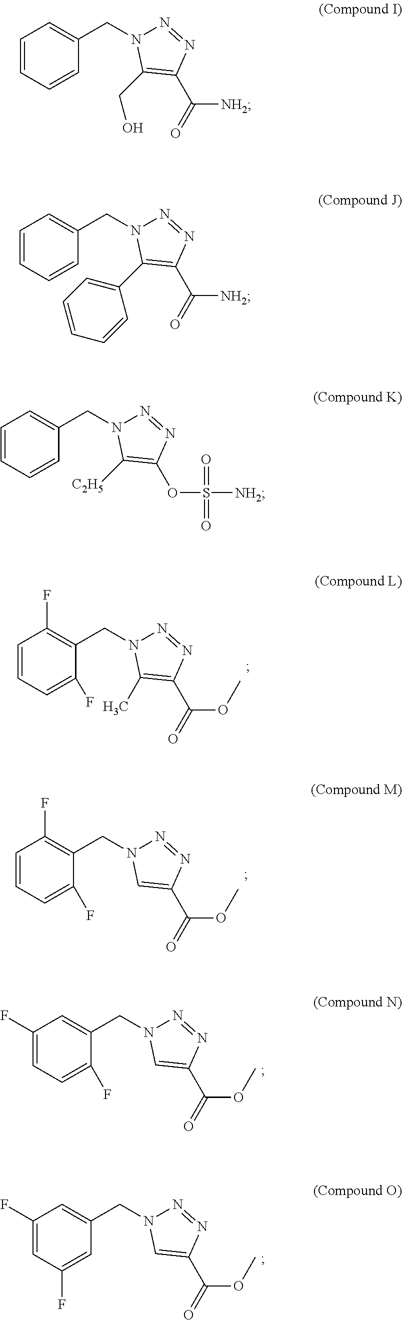 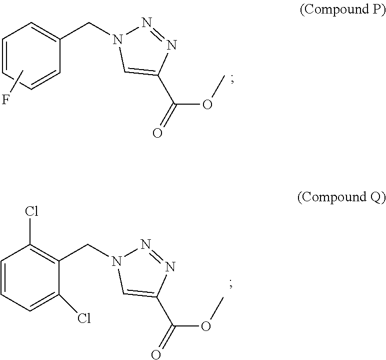
or a salt, solvate, or stereoisomer thereof.

28. The method of claim 26, wherein the one or more Naᵥ1.1 channel blockers of formula I are administered in conjunction with an effective amount of one or more additional biologically active agents.

29. The method of claim 28, wherein the one or more additional biologically active agents comprise enzymes, receptor antagonists or agonists, hormones and antibodies, autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, sympatholytics, sympathomimetics, adrenergic agonist sympathomimetics, intravenous anesthetics, barbiturate intravenous anesthetics, benzodiazepine intravenous anesthetics, opiate agonist intravenous anesthetics, skeletal muscle relaxants, neuromuscular blocker skeletal muscle relaxants, reverse neuromuscular blocker skeletal muscle relaxants, neurological agents, anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, antidepressants, heterocyclic antidepressants, monoamine oxidase inhibitors, selective serotonin re-uptake inhibitors, tricyclic antidepressants, antimanics, anti-psychotics, phenothiazine antipsychotics, anxiolytics, sedatives, hypnotics, barbiturate sedatives, benzodiazepine anxiolytics, sedatives, and hypnotics, and psychostimulants.

30. A method for inhibition of non-inflammatory pain in a subject suffering from a neurological disorder, comprising administering to the subject, an effective amount of a composition comprising one or more one or more Naᵥ1.1 channel blockers of formula I: 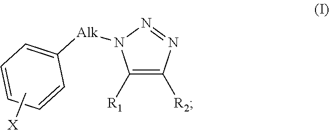
or a salt, solvate, or stereoisomer thereof, wherein X is H, or one or more electron withdrawing groups such as a halogen, NH₂, NO₂, SO₂, CN, or a C₁-C₆ alkyl group; Alk is C₁-C₃ alkyl; R₁ is H, C₁-C₆ alkyl, which may be substituted with OH, NH₂, alkylamino, amido, acyl, sulfonyl, sulfonylamino, and cyano groups.

31. The method of claim 30, wherein the one or more Naᵥ1.1 channel blockers of formula I are selected from the group consisting of: 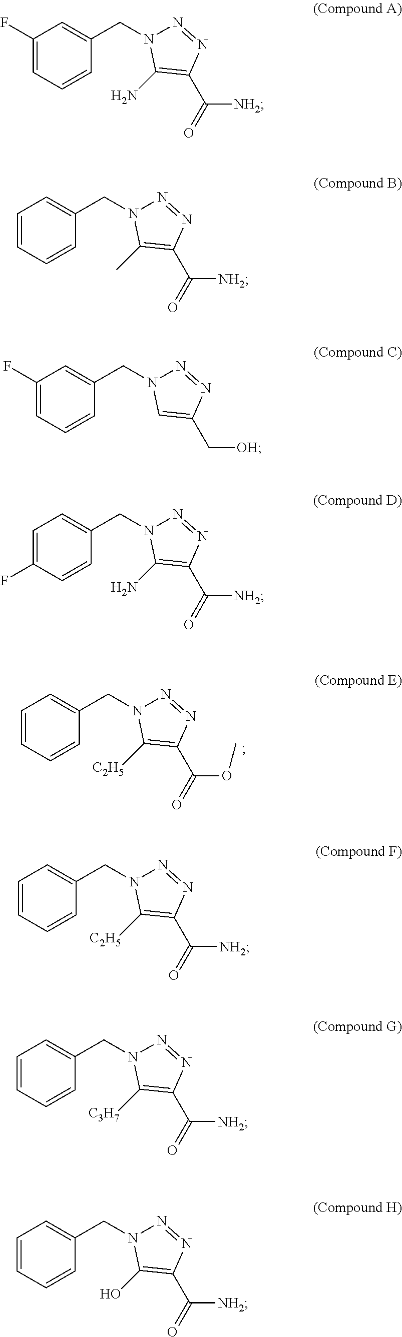 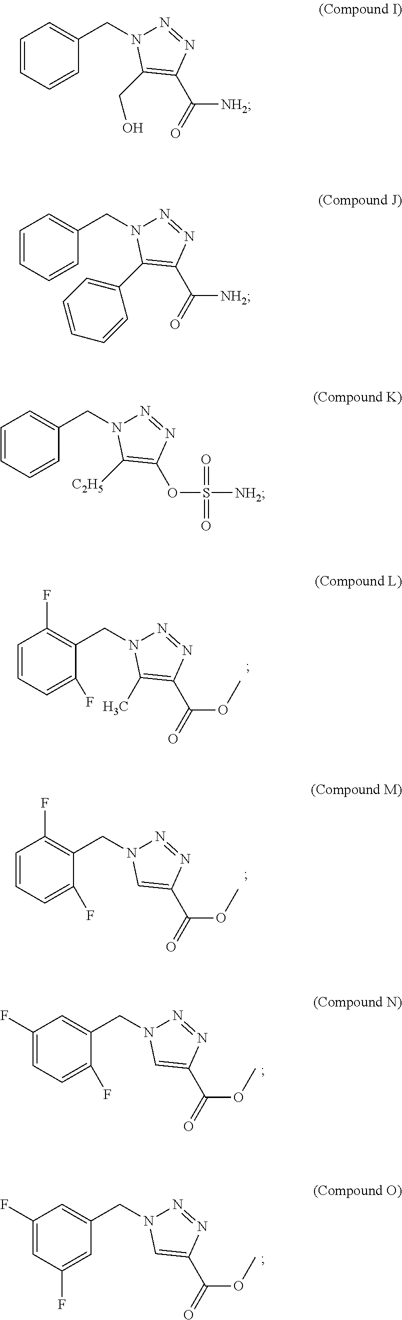 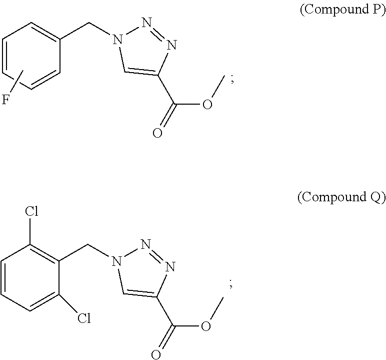
or a salt, solvate, or stereoisomer thereof.

32. The method of claim 30, wherein the one or more Naᵥ1.1 channel blockers of formula I are administered in conjunction with an effective amount of one or more additional biologically active agents.

33. The method of claim 32, wherein the one or more additional biologically active agents comprise enzymes, receptor antagonists or agonists, hormones and antibodies, autonomic agents, such as anticholinergics, antimuscarinic anticholinergics, ergot alkaloids, parasympathomimetics, cholinergic agonist parasympathomimetics, cholinesterase inhibitor parasympathomimetics, sympatholytics, α-blocker sympatholytics, sympatholytics, sympathomimetics, adrenergic agonist sympathomimetics, intravenous anesthetics, barbiturate intravenous anesthetics, benzodiazepine intravenous anesthetics, opiate agonist intravenous anesthetics, skeletal muscle relaxants, neuromuscular blocker skeletal muscle relaxants, reverse neuromuscular blocker skeletal muscle relaxants, neurological agents, anticonvulsants, barbiturate anticonvulsants, benzodiazepine anticonvulsants, anti-migraine agents, anti-parkinsonian agents, anti-vertigo agents, opiate agonists, and opiate antagonists; psychotropic agents, antidepressants, heterocyclic antidepressants, monoamine oxidase inhibitors, selective serotonin re-uptake inhibitors, tricyclic antidepressants, antimanics, anti-psychotics, phenothiazine antipsychotics, anxiolytics, sedatives, hypnotics, barbiturate sedatives, benzodiazepine anxiolytics, sedatives, and hypnotics, and psychostimulants.

34. The method of claim 30, wherein the neurologic disease is selected from the group consisting of: febrile epilepsy, GEFS+, Dravet syndrome (also known as severe myclonic epilepsy of infancy or SMEI), borderline SMEI (SMEB), West syndrome (also known as infantile spasms), Doose syndrome (also known as myoclonic astatic epilepsy), intractable childhood epilepsy with generalized tonic-clonic seizures (ICEGTC), Panayiotopoulos syndrome, familial autism, Rasmussens's encephalitis and Lennox-Gastaut syndrome, Alzheimer's, migraine, including FHM3, the treatment of acute and/or chronic pain associated with mechanosensitive neuronal fibers in disorders including, Irritable Bowel Syndrome, static, mechanical or dynamic allodynias associated with neuropathies, complex regional pain syndrome, postherpetic neuralgia, fibromyalgia, spinal cord injury, menstrual cramps and related diseases.


SEQUENCE LISTING (6 sequences)

SEQ ID NO: 1 - protein, 35 aa, Artificial Sequence
  sythetic sequence
ECRYLFGGCSSTSDCCKHLSCRSDWKYCAWDGTFS

SEQ ID NO: 2 - protein, 34 aa, Artificial Sequence
  synthetic sequence
  MISC_FEATURE (34)..(34): Phe is amidated
ECRYLFGGCKTTADCCKHLGCRTDLYYCAWDGTF

SEQ ID NO: 3 - protein, 34 aa, Artificial Sequence
  synthetic sequence
ECRYLFGGCSSTSDCCKHLSCRSDWKYCAWDGTF

SEQ ID NO: 4 - protein, 34 aa, Artificial Sequence; identical to SEQ ID NO: 2
  synthetic sequence

SEQ ID NO: 5 - DNA, 24 bp, Artificial Sequence
  synthetic sequence
tagcgttcgaacgcactgatttcg

SEQ ID NO: 6 - DNA, 24 bp, Artificial Sequence
  synthetic sequence
cgccgtaaatcaatcgatgagttg